(19) United States
(12) Patent Application Publication
(10) Pub. No.: US 2005/0020680 A1
(43) Pub. Date: Jan. 27, 2005

(54) Hydroxyfattysulfonic acid analogs

(76) Inventors: John R Falck, Dallas, TX (US); Noriyuki Miyata, Toshima-ku (JP); Naoya Ono, Toshima-ku (JP); Tomomichi Chonan, Toshima-ku (JP); Hitomi Hirano, Toshima-ku (JP); Yoshihisa Toda, Toshima-ku (JP); Tohru Tanami, Toshima-ku (JP); Shigeru Okuyama, Toshima-ku (JP)

Correspondence Address: SUGHRUE MION, PLLC, 2100 PENNSYLVANIA AVENUE, N.W., SUITE 800, WASHINGTON, DC, 20037, US

(21) Appl. No.: 10/489,204

(22) PCT Filed: Sep. 9, 2002
(86) PCT No.: PCT/US02/25970

Related U.S. Application Data
(60) Provisional application No. 60/318,874, filed on Sep. 14, 2001.

(51) Int. Cl. A61K 31/185; C 07C 3 9/02

(52) U.S. Cl. 514/553; 562/30

(57) ABSTRACT

A hydroxyfattysulfonic acid analog represented by Formula (I): wherein X is an ethylene group, a vinylene group or an ethynylene group; Y is an ethylene group, a vinylene group, an ethynylene group, OCH₂ or S(O)pCH₂ wherein p is 0, 1 or 2; m is an integer of 1 to 5 inclusive; n is an integer of 0 to 4 inclusive; R¹ is a C₁₋₈ alkyl group, a C₃₋₈ cycloalkyl group, a C₁₋₄ alkyl group substituted with a C₃₋₈ cycloalkyl group, a C₁₋₄ alkyl group substituted with an aryl group or a C₁₋₄ alkyl group substituted with an aryloxy group; R² is a hydrogen atom or a methyl group; R¹ and R² together with the carbon atom to which they are attached may form a C₃₋₈ cycloalkyl group; R³ is a hydrogen atom or a C₂₋₈ acyl group; R⁴ is OR⁵ or NHR⁶, wherein R⁵ is a hydrogen atom, a C₁₋₄ alkyl group, an alkali metal, an alkaline earth metal or an ammonium group and R⁶ is a hydrogen atom or a C₁₋₄ alkyl group; or a pharmaceutically acceptable salt or a hydrate thereof. The compounds of the present invention are useful as an elastase release inhibitor.

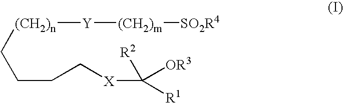

This application is based on and claims priority from U.S. Provisional Patent Application No. 60/318,874, filed Sep. 14, 2001 which is incorporated herein by reference in its entirety for all purposes.

### TECHNICAL FIELD

This invention relates to a novel hydroxyfattysulfonic acid analog having an elastase release-inhibiting activity, a pharmaceutically acceptable salt or a hydrate thereof.

The invention also relates to an elastase release-inhibiting composition which comprises as an active ingredient the hydroxyfattysulfonic acid analog.

### BACKGROUND ART

Protease produced from neutrophils, one of lymphocytes, plays a main role in degrading foreign microorganisms such as bacteria or damaged cells and thus plays an important role in biophylactic reaetion. Neutrophilic elastase, one of serine proteases, (hereinafter simply referred to as elastase) is abundantly released from granules of neutrophils which may develop in the case of infections or inflammatory disorders. Elastase is an enzyme capable of decomposing proteins such as elastin, collagen, proteoglycan, fibronectin, etc., which constitute stroma of in vivo connecting tissues such as lung, cartilage, vascular wall, skin, ligament and so on. Further, it has been elucidated that this enzyme may also act on other proteins or cells.

The elastase maintains homeostasis of a living body, while its action is under control by endogenous inhibitor proteins, typically, α1-protease inhibitor, a 2-macroglobulin, secretory leukocyte protease inhibitor, etc. However, where a balance of elastase and endogenous inhibitor is lost by overproduction of elastase in inflammatory sites or by a lowered inhibitor level, the activity of elastase release may become uncontrollable to cause damage of tissues.

Elastase is known to be involved in pathology of certain diseases such as pulmonary emphysema, respiratory distress syndrome of adults, idiopathic pulmonary fibrosis, cystic pulmonary fibrosis, chronic interstitial pneumonia, chronic bronchitis, chronic sinopulmonary infection, diffuse panbronchiolitis, bronchiectasis, asthma, pancreatitis, nephritis, hepatic insufficiency, chronic rheumatism, arthrosclerosis, osteoarthritis, psoriasis, periodontitis, atherosclerosis, rejection against organ transplantation, premature amniorrhexis, hydroa, shock, sepsis, systemic lupus erythematosus, Crohn's disease, disseminated intravenous coagulation, cerebral infarction, cardiac disorders, ischemic reperfusion disorders observed in renal diseases, cicatrization of corneal tissues, spondylitis, and etc.

In view of the foregoing, an elastase release inhibitor is useful as a therapeutic or preventive agent for these diseases. Extensive studies have recently been made with expectation and various elastase release inhibitors have been reported. However, their activity is not quite satisfactory. Moreover, any clinically useful drug has not yet been found out as an elastase release-inhibiting agent comprising a hydroxyfattysulfonic acid analog.

### DISCLOSURE OF INVENTION

It is an object of this invention to provide a novel compound having a prominent elastase release-inhibiting activity.

It is another object of this invention to provide an elastase release-inhibiting composition which comprises the hydroxyfattysulfonic acid analog or a pharmaceutically acceptable salt or hydrate thereof and a pharmaceutically acceptable carrier.

### BRIEF DESCRIPTION OF DRAWINGS

FIG. 1 represents an effect of compound 33 on infarct volume in rat t-MCAo model.

The infract volumes of total (open column), cortex (closed column) and subcortex (hatched column) were determined 71 hrs after reperfusion. Data are presented as mean±SEM. *p<0.05 vs vehicle-treated group (Dunnett's test).

### DETAILED DESCRIPTION

The present inventors studied intensively to find that a novel hydroxyfattysulfonic acid analog represented by the following formula shows an elastase release-inhibiting activity, upon which this invention has been completed.

More specifically, the invention is directed to a hydroxyfattysulfonic acid analog represented by the following formula (I): 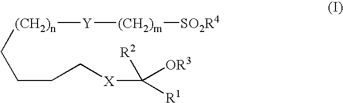
wherein
X represents an ethylene group, a vinylene group or an ethynylene group;
Y represents an ethylene group, a vinylene group, an ethynylene group, OCH₂ or S(O)pCH₂, wherein p is 0, 1 or 2;
m represents an integer of 1 to 5 inclusive;
n represents an integer of 0 to 4 inclusive;
   R¹ represents a C₁₋₈ alkyl group, a C₃₋₈ cycloalkyl group, a C₁₋₄ alkyl group substituted with a C₃₋₈ cycloalkyl group, a C₁₋₄ alkyl group substituted with an aryl group or a C₁₋₄ alkyl group substituted with an aryloxy group;
   R² represents a hydrogen atom or a methyl group;
R¹ and R² together with the carbon atom to which they are attached may form a C₃₋₈ cycloalkyl group;
   R³ represents a hydrogen atom or a C₂₋₈ acyl group;
   R⁴ represents OR⁵ or NHR⁶, wherein R⁵ represents a hydrogen atom, a C₁₋₄ alkyl group, an alkali metal, an alkaline earth metal or an ammonium group and R⁶ represents a hydrogen atom or a C₁₋₄ alkyl group, or a pharmaceutically acceptable salt or a hydrate thereof. Especially preferred compounds are sodium (R)-(4Z, 13Z)-15-hydroxynonadeca-4,13-diene -1-sulfonate and sodium (R)-(Z)-15-hydroxynonadec-13-ene -1-sulfonate.

As used herein, the term “vinylene group” means a cis-vinylene or a trans-vinylene group.

As used herein, the term “C₁₋₄ alkyl group” means a straight or branched alkyl group, which includes, for example, a methyl group, an ethyl group, a propyl group, an isopropyl group, a butyl group and an isobutyl group.

As used herein, the term “C₁₋₈ alkyl group” means a straight or branched alkyl group, which includes, for example, a methyl group, an ethyl group, a propyl group, a butyl group, an isobutyl group, a pentyl group, a hexyl group, a heptyl group, an octyl group, a 2-methylhex-1-yl group and a 2,4-dimethylpent-1-yl group.

As used herein, the “C₃₋₈ cycloalkyl group” includes, for example, a cyclopropyl group, a cyclobutyl group, a cyclopentyl group, a cyclohexyl group, a cycloheptyl group and a cyclooctyl group.

The symbol m represents an integer of 1-5 inclusive, and the symbol n represents an integer of 0-4 inclusive.

The sum of m and n is preferably and integer 4 to 8.

As used herein, the term “C₁₋₄ alkyl group substituted with an aryl group” includes, for example, a benzyl group, a methoxybenzyl group, a phenethyl group, phenylpropyl group, a 2-phenylprop-2-yl group, a 3-phenylbut-1-yl group and a tolylmethyl group.

As used herein, the term “a C₁₋₄ alkyl group substituted with a C₃₋₈ cycloalkyl group” includes, for example, a cyclopentylmethyl group, a cyclohexylmethyl group, a cyclohexylethyl group, a cyclopropylethyl group and a cycloheptylpropyl group.

As used herein, the term “C₁₋₄ alkyl group substituted with an aryloxy group” includes, for example, a phenoxymethyl group, a phenoxyethyl group, phenoxypropyl group, a 2-phenoxyprop-2-yl group and a tolyloxymethyl group.

As used herein, the “C₂₋₈ acyl group” includes, for example, an acetyl group, a propionyl group, a butyryl group, an isobutyryl group, a valeryl group, a pivaloyl group, a benzoyl group and a toluoyl group.

As used herein, “an alkali metal” includes, for example, lithium, sodium and potassium.

As used herein, “an alkaline earth metal” includes, for example, calcium and magnesium.

As used herein, “an ammonium group” includes, for example, salts with ammonia, methylamine, dimethylamine, diethylamine, cyclopentylamine, benzylamine, piperidine, monoethanolamine, diethanolamine, monomethyl-monoethanolamine, triethanolamine, toromethamine, lysine, ornithine, piperazine, benzathine, aminopyridine, procaine, choline, a tetra-alkyl-ammonium, tris(hydroxymethyl)aminomethane and ethylenediamine.

The compounds of the formula (I) can be prepared, for example, by the processes as shown in the following Reaction schemes.

In the Reaction Schemes, Z and Z² may be the same or different and each represents a halogen atom or a leaving group such as a methanesulfonyloxy group and a p-toluenesulfonyloxy group; Y² represents a OCH₂ group and a SCH₂ group; Y³ represents an ethylene group, a vinylene group, an ethynylene group, a OCH₂ group and a SCH₂ group; Y⁴ represents an ethylene group, a cis-vinylene group, a OCH₂ group and a SCH₂ group; X² represents a vinylene group and an ethynylene group; X³ represents an ethylene group and a cis-vinylene group; R⁷ and R⁸ may be the same or different and each represents a protecting group for hydroxyl group, which is stable to a base, such as a trimethylsilyl group, a triethylsilyl group, a tert-butyldimethylsilyl group, a tert-butyldiphenylsilyl group, a methoxymethyl group, an ethoxyethyl group, a tetrahydropyranyl group, a benzyl group and a p-methoxybenzyl group; R³¹ is the same as R³ excluding the hydrogen atom; R⁵¹ represents a C₁₋₄ alkyl group; p1 is an integer of 1 or 2; and R¹, R², R³, R⁴, R⁶, X, Y, m, n and p are as defined above.

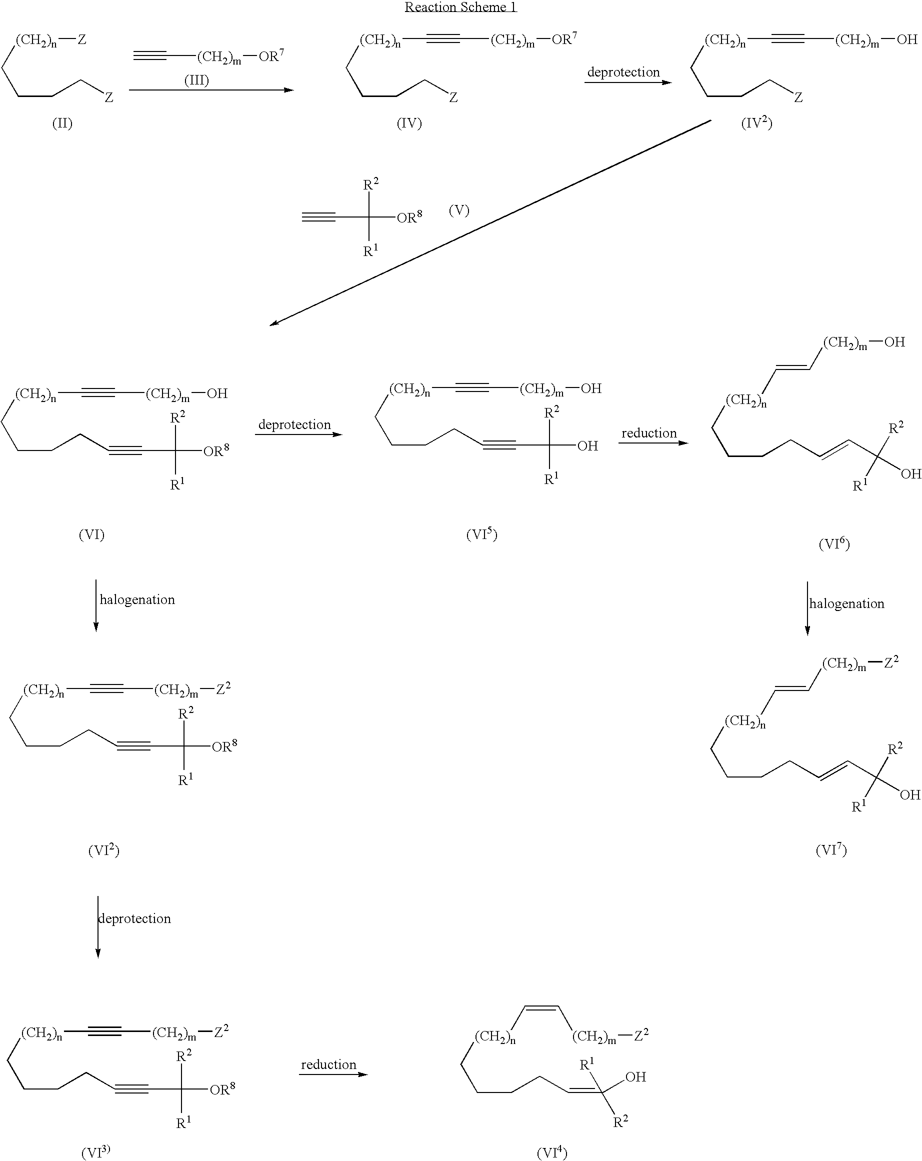

(1) A compound of the formula (II) is reacted with a compound of the formula (III) in a suitable organic solvent such as tetrahydrofuran, hexamethylphosphoric triamide, N,N′-dimethylpropyleneurea, NH₃, dimethyl sulfoxide or N,N-dimethylformamide, or a mixture thereof, in the presence of a base such as n-BuLi, LiNH₂ or NaNH₂ at a temperature of −78° C. to room temperature to give a compound of the formula (IV).

(2) A compound of the formula (IV) is treated with an organic acid such as p-toluenesulfonic acid or acetic acid, or an amine salt thereof such as pyridinium p-toluenesulfonate, or an inorganic acid such as hydrochloric acid or sulfuric acid, in a suitable organic solvent such as an alcohol solvent, e.g., MeOH or EtOH, or an ether solvent, e.g., tetrahydrofuran or diethyl ether, or a mixture thereof, at a temperature of 0° C. to 60° C., preferably from room temperature to 40° C., thereby removing the protecting group for the hydroxyl group to give a compound of the formula (IV²).

(3) A compound of the formula (IV²) and a compound of the formula (V) are reacted in the same manner as in the above (1) to give a compound of the formula (VI).

(4) A compound of the formula (VI) is halogenated directly using CCl₄—PPh₃, PBr₃, CBr₄—PPh₃, I₂—PPh₃ or the like, or conversion to leaving group using methansulfonyl chloride, p-toluenesulfonyl chloride or the like, to give a compound the formula (VI²).

(5) A compound of the (VI) or (VI²) is reacted in the same manner as in the above (2) to give a compound of the formula (VI⁵) or (VI³), respectively.

(6) A compound of the formula (VI³) is reduced, for example, by a method using a Pd-containing catalyst, e.g., Pd—CaCO₃, Pd(OAc)₂ or a Ni-containing catalyst, e.g., Ni(OAc)₂ and NaBH₄ under hydrogen atmosphere, and where necessary, further adding ethylenediamine, quinoline or the like, a method using Zn as a reducing agent in MeOH or AcOH and others to give a compound of the formula (VI⁴).

(7) A compound of the formula (VI⁵) is reduced, for example, by method using a hydride reduction, e.g., LAH (lithium aluminum hydride, Red-Al (sodium bis(2-methoxyethoxy)aluminum hydride) in diethyl ether, tetrahydrofuran, DME(ethylene glycol dimethyl ether) or toluene and others or a dissolving-metal reduction, e.g., Li-liquid NH₃ or Na-liquid NH₃ to give a compound of the formula (VI⁶).

(8) A compound of the formula (VI⁶) is reacted in the same manner as in the above (4) to give a compound of the formula (VI⁷).

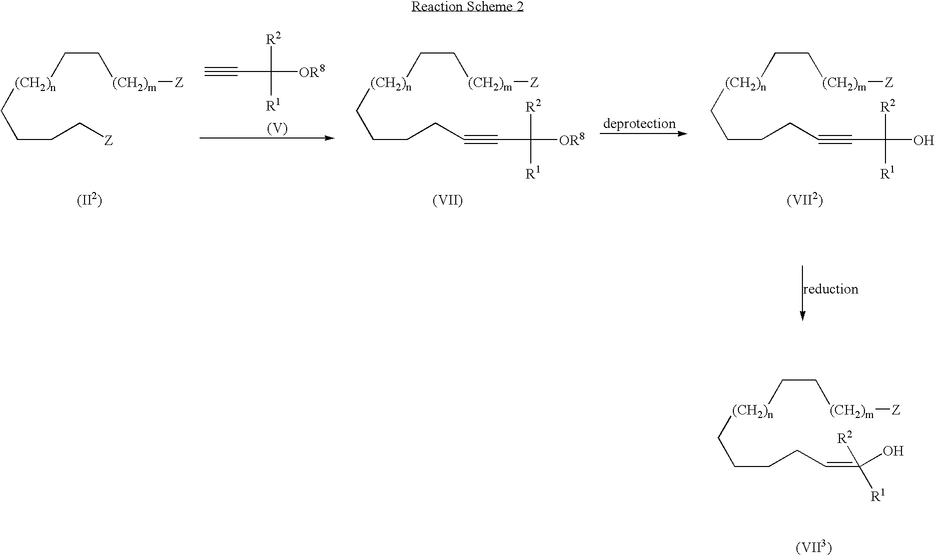

(9) A compound of the formula (II²) is reacted with a compound of the formula (V) in the same manner as in the above (1) to give a compound of the formula (VII).

(10) A compound of the formula (VII) is reacted in the same manner as in the above (2) to give a compound of the formula (VII²).

(11) A compound of the formula (VII²) is reduced in the same manner as in the above (6) to give a compound of the formula (VII³).

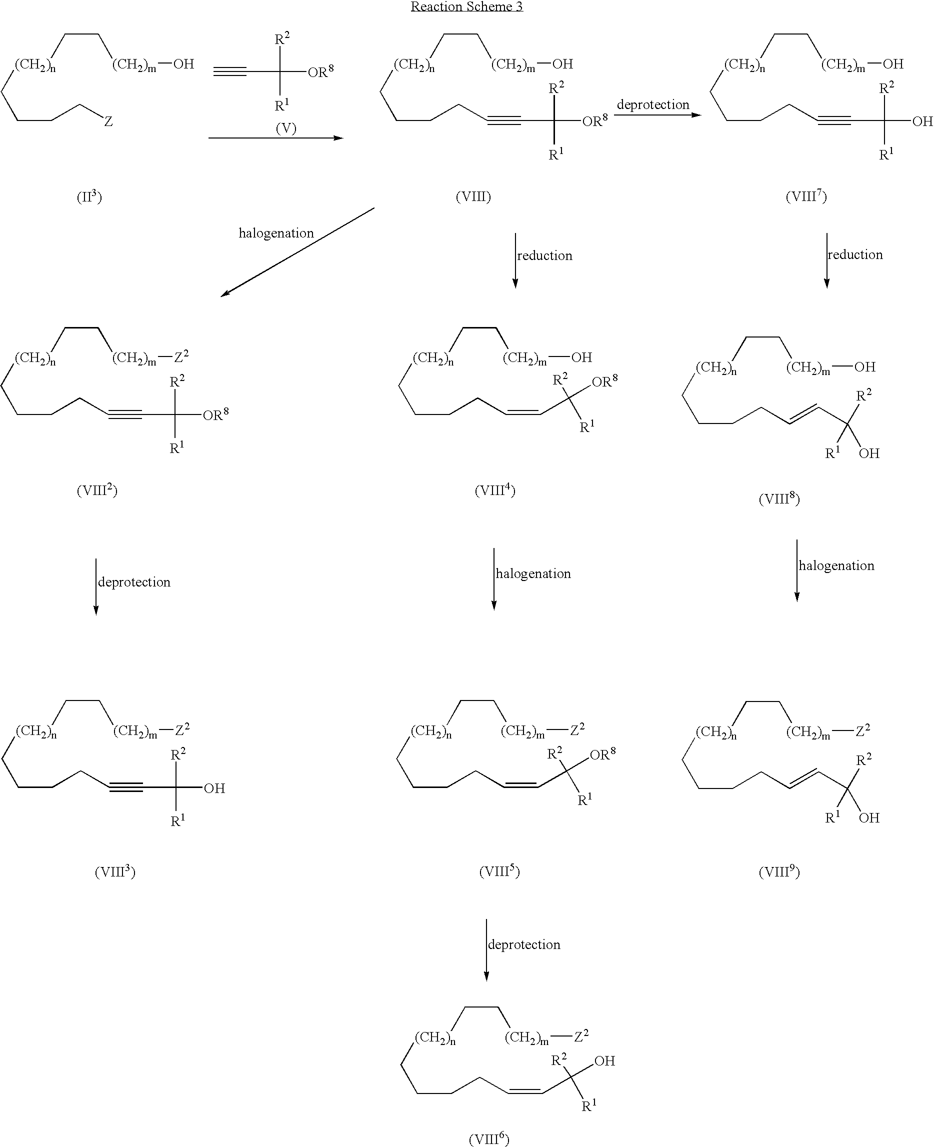

(12) A compound of the formula (II³) is reacted with a compound of the formula (V) in the same manner as in the above (1) to give a compound of the formula (VIII).

(13) A compound of the formula (VIII) is reduced in the same manner as in the above (6) to give a compound of the formula (VIII⁴).

(14) A compound of the formula (VIII) is reacted in the same manner as in the above (2) to give a compound of the formula (VIII⁷).

(15) A compound of the formula (VIII⁷) is reduced in the same manner as in the above (7) to give a compound of the formula (VIII⁸).

(16) A compound of the formula (VIII), (VIII⁴) or (VIII⁸) is reacted in the same manner as in the above (4) to give a compound of the formula (VIII²), (VIII⁵) or (VIII⁹), respectively.

(17) A compound of the formula (VIII²) or (VIII⁵) is reacted in the same manner as in the above (2) to give a compound of the formula (VIII³) or (VIII⁶), respectively.

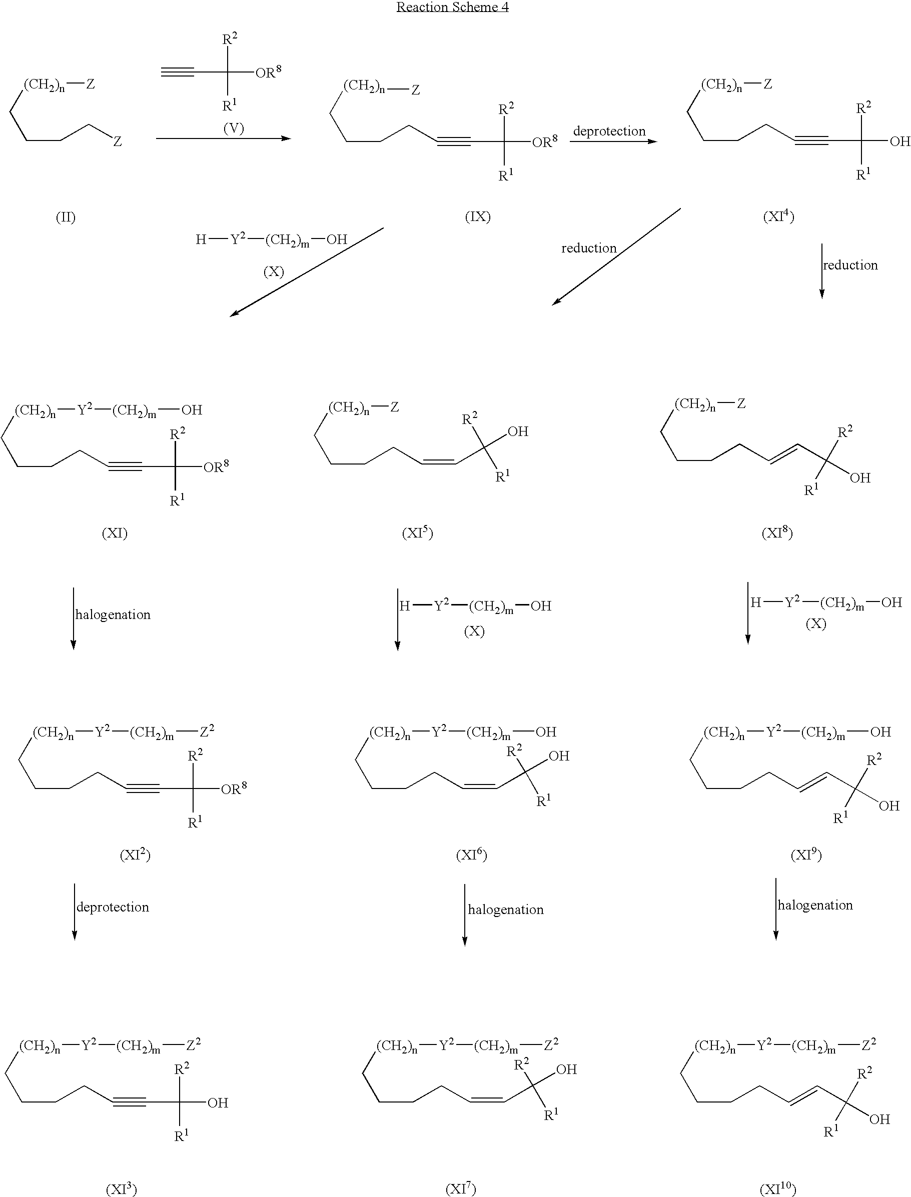

(18) A compound of the formula (II) is reacted with a compound of the formula (V) in the same manner as in the above (1) to give a compound of the formula (IX).

(19) A compound of the formula (IX) is reacted in the same manner as in the above (2) to give a compound of the formula (XI⁴).

(20) A compound of the formula (XI⁴) is reduced in the same manner as in the above (6) to give a compound of the formula (XI⁵).

(21) A compound of the formula (XI⁴) is reduced in the same manner as in the above (7) to give a compound of the formula (XI⁸).

(22) A compound of the formula (IX), (XI⁵) or (XI⁸) is reacted with a compound of the formula (X) in a suitable organic solvent such as MeOH, EtOH, tert-BuOH, acetone, N,N-dimethylformamide, tetrahydrofuran or acetonitrile, in the presence of a suitable base such as Et₃N, NaH, KH, NaHCO₃, K₂CO₃, NaOH, CaCO₃ or quaternary ammonium salt (e.g., Et₄NBr) and, where necessary, further adding NaI or the like, to give a compound of the formula (XI), (XI⁶) or (XI⁹), respectively.

(23) A compound of the formula (XI), (XI⁶) or (XI⁹) is halogenated in the same manner as in the above (4) to give a compound of the formula (XI²), (XI⁷) or (XI¹⁰), respectively.

(24) A compound of the formula (XI²) is reacted in the same manner as in the above (2) to give a compound of the formula (XI³).

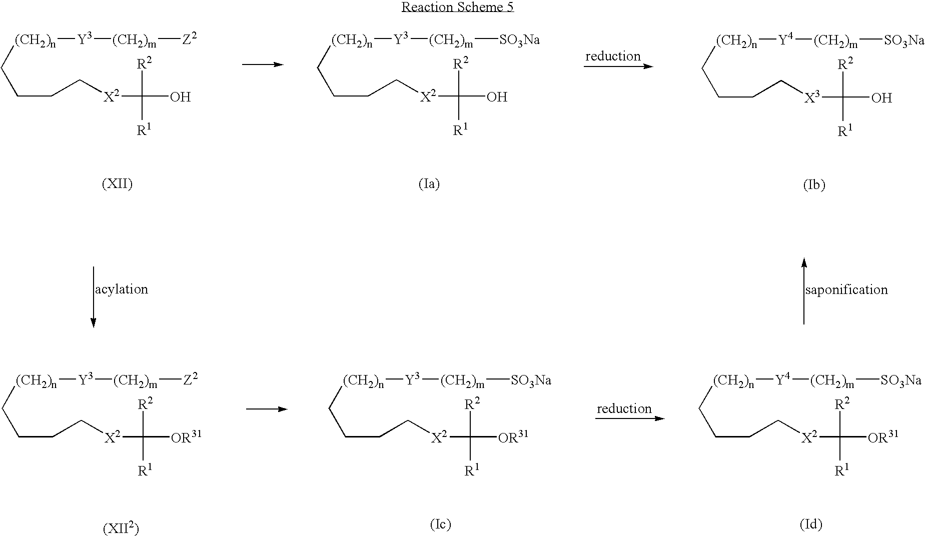

(25) A compound of the formula (XII) is reacted with a acid anhydride such as acetic anhydride, butyric anhydride, pivalic anhydride, valeric anhydride or the like, or a acid chloride such as acetyl chloride, pivaloyl chloride, valeryl chloride, benzoyl chloride, toluoyl chloride or the like in a suitable organic solvent such as pyridine or dichloromethane, and where necessary, in the presence of an additive such as 4-(dimethylamino)pyridine or the like, to give a compound of the formula (XII²).

(26) A compound of the formula (XII) or (XII²) is reacted with sodium sulfite in a suitable mixed solvent with water, such as dimethyl sulfoxide, N,N-dimethylformamide, tetrahydrofuran, dioxane, MeOH, EtOH or acetone, and where necessary, in the presence of an additive such as NaI, to give a compound of the formula (Ia) or (Ic), respectively.

(27) A compound of the formula (Ia) or (Ic) is reduced, for example, by a method using a Pd-containing catalyst, e.g., Pd-carbon, Pd—CaCO₃, Pd(OAc)₂ under hydrogen to give a compound of the formula (Ib) or (Id), respectively.

(28) A compound of the formula (Id) is treated with a base conventionally employed for hydrolysis such as NaOMe, NaOEt or NaOH, in a suitable organic solvent such as MeOH, EtOH, dioxane or water, or a mixture thereof to give a compound of the formula (Ib).

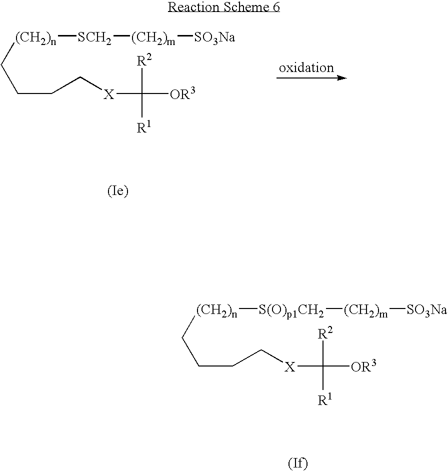

(29) A compound of the formula (Ie) is treated with an oxidizing agent such as NaIO₄ in a suitable solvent such as water, MeOH or EtOH, at a temperature of −20° C. to reflux, to give a compound of the formula (If).

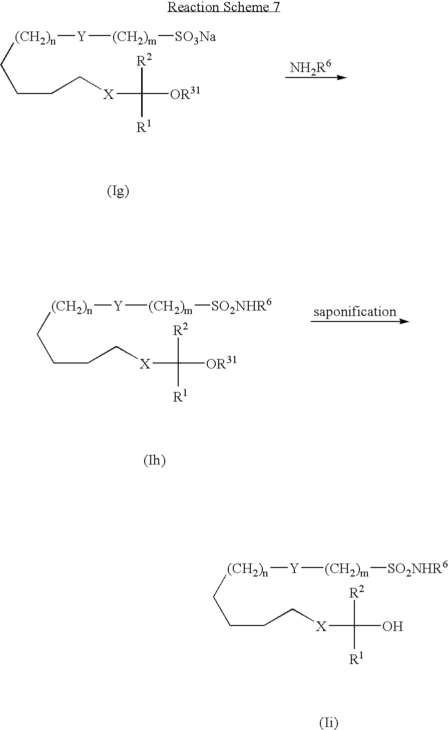

(30) A compound of the formula (Ig) is reacted with SOCl₂, PCl₃ or PCl₅ in a suitable organic solvent, such as dimethyl sulfoxide or N,N-dimethylformamide, followed by reaction with NH₂R⁶ to give a compound of the formula (Ih).

(31) A compound of the formula (Ih) is reacted in the same manner as in the above (28) to give a compound of the formula (Ii).

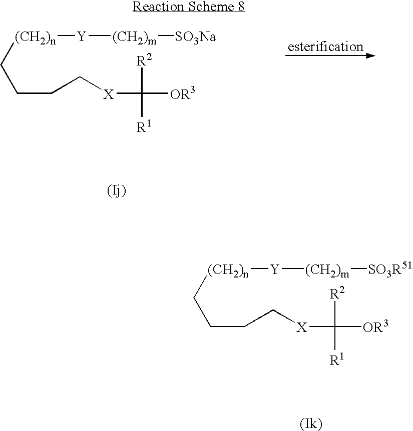

(32) A compound of the formula (Ij) is reacted with hydrochloric acid or sulfuric acid in a suitable solvent, such as MeOH, EtOH or dioxane, followed by treatment with diazoalkane such as diazomethane, diazoethane, diazopropane or (trimethylsilyl)diazomethane to give a compound of the formula (Ik).

The present compounds may be administered systemically or orally via oral or parenteral, such as rectal, subcutaneous, intermuscular, intravenous, transdermal and nasal/lung inhalation or percutaneous route. They can be administered orally in the dosage form of tablets, powders, granules, fine powders, capsules, solutions, emulsions, suspensions or the like as prepared in a conventional manner. A pharmaceutical preparation for intravenous route may be in the form of aqueous or non-aqueous solutions, emulsions, suspensions, solid preparations to be used after dissolving in an injectable solvent immediately before application, or the like. The compounds of the invention may be formulated into a pharmaceutical preparation by forming an inclusion compound with α-, β- or γ-cyclodextrin or substituted cyclodextrin.

Also, aqueous or non-aqueous solutions, emulsions or suspensions of the compounds may be administered, for example, via injection. A dose may be varied depending on the age, body weight and other factors of patients, and 1 ng/kg/day-1000 mg/kg/day is given to adults once a day or in several divided forms.

Representative compounds represented by the formula (I) will be illustrated below:

| Compound | R¹ | R² | R³ | X | Y | m | n | R⁴ | * |
|---|---|---|---|---|---|---|---|---|---|
| 1 | nOct | H | H | C≡C | C≡C | 5 | 4 | OLi | S |
| 2 | nPen | H | Tolu | C≡C | C≡C | 5 | 4 | ONa | S |
| 3 | nBu | H | H | C≡C | C≡C | 4 | 3 | ONa | R |
| 4 | nBu | H | H | C≡C | C≡C | 3 | 3 | OK | R |
| 5 | nBu | Me | H | C≡C | C≡C | 3 | 3 | OH.NH₃ | RS |
| 6 | nPr | H | H | C≡C | C≡C | 3 | 3 | O.1/2.Ca | R |
| 7 | nPen | H | H | C≡C | CH₂CH₂ | 2 | 3 | ONa | R |
| 8 | nPen | Me | H | C≡C | CH₂CH₂ | 3 | 3 | ONa | RS |
| 9 | nBu | H | H | C≡C | CH₂CH₂ | 5 | 3 | ONa | RS |
| 10 | nBu | H | H | C≡C | CH₂CH₂ | 3 | 3 | ONa | R |
| 11 | nBu | H | H | C≡C | CH₂CH₂ | 1 | 0 | ONa | R |
| 12 | iBu | H | H | C≡C | CH₂CH₂ | 3 | 3 | ONa | RS |
| 13 | cHex | H | H | C≡C | CH₂CH₂ | 3 | 3 | ONa | S |
| 14 | cPr | H | H | C≡C | CH₂CH₂ | 5 | 3 | NHCH₃ | R |
| 15 | Bn | H | H | C≡C | CH₂CH₂ | 3 | 3 | ONa | S |
| 16 | Phen | H | H | C≡C | CH₂CH₂ | 1 | 0 | ONa | R |
| 17 | PhOCH₂ | H | H | C≡C | CH₂CH₂ | 3 | 3 | ONa | R |
| 18 | —(CH₂)₄— | | H | C≡C | CH₂CH₂ | 3 | 3 | ONa | |
| 19 | nBu | H | H | C≡C | SCH₂ | 2 | 3 | ONa | R |
| 20 | nBu | H | H | C≡C | S(O)CH₂ | 2 | 3 | ONa | R |
| 21 | nBu | H | H | C≡C | OCH₂ | 2 | 3 | ONa | R |
| 22 | nHep | H | H | (Z)CH═CH | (Z)CH═CH | 1 | 3 | OK | R |
| 23 | nBu | H | H | (Z)CH═CH | (Z)CH═CH | 3 | 3 | ONa | R |
| 24 | Et | H | H | (Z)CH═CH | (Z)CH═CH | 4 | 1 | O.1/2.Mg | S |
| 25 | nBu | H | H | (E)CH═CH | (E)CH═CH | 3 | 3 | ONa | R |
| 26 | —(CH₂)₅— | | H | (Z)CH═CH | (Z)CH═CH | 3 | 3 | ONa | |
| 27 | nHex | H | H | (Z)CH═CH | (Z)CH═CH | 3 | 3 | OH.tris | R |
| 28 | nPen | Me | H | (Z)CH═CH | CH₂CH₂ | 1 | 3 | ONa | RS |
| 29 | nPen | H | H | (Z)CH═CH | CH₂CH₂ | 2 | 3 | ONa | R |
| 30 | nBu | H | H | (Z)CH═CH | CH₂CH₂ | 4 | 3 | ONa | R |
| 31 | nBu | H | Ac | (Z)CH═CH | CH₂CH₂ | 3 | 3 | ONa | R |
| 32 | nBu | H | Bz | (Z)CH═CH | CH₂CH₂ | 3 | 3 | ONa | R |
| 33 | nBu | H | H | (Z)CH═CH | CH₂CH₂ | 3 | 3 | ONa | R |
| 34 | nBu | H | H | (Z)CH═CH | CH₂CH₂ | 3 | 3 | ONa | S |
| 35 | nBu | H | H | (Z)CH═CH | CH₂CH₂ | 3 | 3 | OK | R |
| 36 | nBu | H | H | (Z)CH═CH | CH₂CH₂ | 3 | 3 | O.1/2.Ca | R |
| 37 | nBu | H | H | (Z)CH═CH | CH₂CH₂ | 3 | 3 | OLi | R |
| 38 | nBu | H | H | (Z)CH═CH | CH₂CH₂ | 3 | 3 | OH.NH₃ | R |
| 39 | nBu | H | H | (Z)CH═CH | CH₂CH₂ | 3 | 3 | OH.tris | R |
| 40 | nBu | H | H | (Z)CH═CH | CH₂CH₂ | 3 | 3 | OH-(L)Lys | R |
| 41 | nBu | H | H | (Z)CH═CH | CH₂CH₂ | 1 | 3 | ONa | R |
| 42 | nBu | H | H | (Z)CH═CH | CH₂CH₂ | 2 | 3 | ONa | R |
| 43 | nBu | H | H | (E)CH═CH | CH₂CH₂ | 3 | 3 | ONa | R |
| 44 | nBu | H | H | (E)CH═CH | CH₂CH₂ | 3 | 3 | ONa | S |
| 45 | nBu | H | Ac | (Z)CH═CH | CH₂CH₂ | 3 | 3 | NH₂ | R |
| 46 | nBu | H | H | (Z)CH═CH | CH₂CH₂ | 3 | 3 | NH₂ | R |
| 47 | nBu | H | H | (Z)CH═CH | SCH₂ | 2 | 3 | ONa | R |
| 48 | nBu | H | H | (Z)CH═CH | OCH₂ | 2 | 3 | ONa | R |
| 49 | nBu | H | Piva | (E)CH═CH | OCH₂ | 2 | 3 | ONa | R |
| 50 | —(CH₂)₃— | | H | (E)CH═CH | CH₂CH₂ | 3 | 3 | ONa | |
| 51 | nOct | H | H | CH₂CH₂ | CH₂CH₂ | 3 | 3 | OH.NH₃ | R |
| 52 | nPen | Me | H | CH₂CH₂ | CH₂CH₂ | 3 | 3 | OH.NH₂Me | RS |
| 53 | nBu | H | H | CH₂CH₂ | CH₂CH₂ | 3 | 3 | ONa | R |
| 54 | nBu | H | Vale | CH₂CH₂ | CH₂CH₂ | 3 | 3 | ONa | R |
| 55 | nBu | H | Ac | CH₂CH₂ | CH₂CH₂ | 3 | 3 | NH-nPr | R |
| 56 | nBu | H | H | CH₂CH₂ | CH₂CH₂ | 3 | 3 | NH-nPr | R |
| 57 | nBu | H | Ac | CH₂CH₂ | CH₂CH₂ | 3 | 3 | NH₂ | R |
| 58 | nBu | H | H | CH₂CH₂ | CH₂CH₂ | 3 | 3 | NH₂ | R |
| 59 | nBu | H | H | CH₂CH₂ | SCH₂ | 3 | 3 | OH-pri | R |
| 60 | nBu | H | H | CH₂CH₂ | S(O)CH₂ | 3 | 3 | OK | R |
| 61 | nBu | H | H | CH₂CH₂ | S(O)₂CH₂ | 3 | 3 | OK | R |
| 62 | —(CH₂)₄— | | H | CH₂CH₂ | SCH₂ | 5 | 4 | ONa | |
| 63 | —(CH₂)₄— | | H | CH₂CH₂ | OCH₂ | 5 | 4 | NHEt | |
| 64 | Me | H | H | CH₂CH₂ | OCH₂ | 5 | 4 | OH-1/2.pra | R |
| 65 | —(CH₂)₂— | | H | CH₂CH₂ | OCH₂ | 5 | 4 | ONa | |
| 66 | nBu | H | H | C≡C | C≡C | 3 | 3 | OMe | R |
| 67 | nBu | H | H | C≡C | C≡C | 4 | 3 | OMe | R |
| 68 | nBu | H | H | C≡C | CH₂CH₂ | 3 | 3 | OMe | R |
| 69 | —(CH₂)₃— | | H | C≡C | CH₂CH₂ | 3 | 3 | O-nPr | |
| 70 | nBu | H | H | (Z)CH═CH | (Z)CH═CH | 4 | 3 | O-nBu | R |
| 71 | nBu | H | H | (Z)CH═CH | (Z)CH═CH | 3 | 3 | OMe | R |
| 72 | nBu | H | H | (Z)CH═CH | CH₂CH₂ | 3 | 3 | OMe | R |
| 73 | nBu | H | Ac | (Z)CH═CH | CH₂CH₂ | 3 | 3 | OEt | R |
| 74 | nBu | H | H | CH₂CH₂ | CH₂CH₂ | 3 | 3 | OEt | R |
| 75 | cPenCH₂ | H | H | CH₂CH₂ | CH₂CH₂ | 3 | 3 | OEt | S |

Ac: acetyl,
Bn: benzyl,
iBu: iso-butyl,
nBu: n-butyl,
Bz: benzoyl,
Et: ethyl,
cHex: cyclohexyl,
nOct: n-octyl,
cPen: cyclopentyl,
nPen: n-pentyl,
Ph: phenyl,
Phen: phenetyl,
Piva: pivaloyl,
nPr: n-propyl,
cPr: cyclopropyl,
Tolu: toluoyl,
Vale: valeryl,
tris: NH₂C(CH₂OH)₃,
(L)Lys; L-Lysine,
pra: piperazine,
pri: piperidine
*: Absolute configuration for carbon atom to which R¹ and R² are attached

The present compounds have a potent elastase release-inhibiting activity and are therefore useful for the treatment and prevention of diseases in which elastase is involved.

### BEST MODE FOR CARRYING OUT THE INVENTION

### EXAMPLE

This invention will be more specifically illustrated by way of the following Examples and Test Examples.

### Example 1

Sodium (R)-(4Z, 13Z)-15-hydroxynonadeca-1,13-diene -1-sulfonate (Compound No. 23)

(1) n-BuLi (13.4 mL, 2.66M in hexane, 35.6 mmol) was added dropwise at −10° C., under argon stream, to a solution of 5-tetrahydropyranyloxy-1-pentyne (5.0 g, 29.7 mmol) in THF (tetrahydrofuran) (30 mL). Thereafter, the reaction solution was stirred at that temperature for 30 minutes. The reaction solution was added dropwise to a solution of 1,7-dibromoheptane (15.32 g, 59.41 mmol) in a mixed solvent of THF (100 mL) and DMPU (N,N′-dimethylpropyleneurea) (10 mL) at 0° C. Thereafter, the reaction solution was stirred at 0° C. for 1 hour and then stirred at room temperature for 1 hour. To the resulting solution was added aqueous hydrochloric acid (20 mL, 3.0M) and the mixture was extracted with AcOEt (150 mL×2). The organic layer was washed with brine (500 mL), dried over anhydrous magnesium sulfate and concentrated. The resulting crude product was purified by silica gel column chromatography to afford 2-(12-bromododec-4-ynyloxy)tetrahydropyran (9.51 g).

¹H-NMR (CDCl₃, 300 MHz) δ ppm: 1.20-1.63 (m, 12H), 1.64-1.92 (m, 6H), 2.09-2.17 (m, 2H), 2.20-2.30 (m, 2H), 3.41 (t, J=6.8 Hz, 2H), 3.44-3.55 (m, 2H), 3.77-3.92 (m, 2H), 4.57-4.63 (m, 1H)

IR (neat): 3400, 2934, 2857, 1440, 1384, 1354, 1200, 1260, 1138, 1120, 1034, 1063, 990, 902, 869, 815, 646, 563 cm⁻¹

(2) Aqueous hydrochloric acid (0.58 mL, 3.0M) was added at room temperature to a solution of the compound obtained in the above (1) (7.0 g, 20.3 mmol) in MeOH (29 mL), and the mixture was stirred at room temperature overnight. To the reaction solution was added a saturated aqueous NaHCO₃ and then the mixture was extracted with AcOEt (100 mL). The organic layer was washed with brine, dried over anhydrous magnesium sulfate and concentrated. The resulting crude product was purified by silica gel column chromatography to afford 12-bromododec-4-yn-1-ol (4.75 g) To a solution of that compound (3.96 g, 15 mmol) and (R)-3-tert-butyldimethylsilanyloxy-1-heptyne (3.82 g, 16.9 mmol) in a mixed solvent of THF (169 mL) and HMPA (hexamethylphosphoric triamide) (67.6 mL) was added dropwise n-BuLi (16.8 mL, 2.66M in hexane, 44.6 mmol) at −60° C. under argon stream. Thereafter, the temperature of the reaction solution was allowed to rise up to 0° C. over about 3.5 hours. To the resulting solution was added water and the mixture was extracted with ACOEt (200 mL×2). The organic layer was washed with aqueous hydrochloric acid (20 mL, 3.0M), water and brine, dried over anhydrous magnesium sulfate and concentrated. The resulting crude product was purified by silica gel column chromatography to afford (R)-15-(tert-butyldimethylsilanyloxy)nonadeca-4,13-diyn-1-ol (6.38 g).

¹H-NMR (CDCl₃, 300 MHZ) δ ppm: 0.10 (s, 3H), 0.12 (s, 3H), 0.84-0.97 (m, 12H), 1.23-1.58 (m, 14H), 1.59-1.68 (m, 2H), 1.69-1.80 (m, 2H), 2.10-2.22 (m, 4H), 2.25-2.32 (m, 2H), 3.76 (t, J=6.0 Hz, 2H), 4.28-4.35 (m, 1H)

IR (neat): 3368, 2931, 2858, 2360, 1712, 1463, 1385, 1361, 1337, 1251, 1152, 1078, 937, 838, 778, 669, 424 cm⁻¹

(3) A solution of triphenylphosphine(2.20 g, 9.73 mmol) in CH₂Cl₂ (dichloromethane)(10 mL) was added at 0° C. to a solution of the compound obtained in the above (2) (2.73 g, 6.95 mmol) and carbon tetrabromide (3.0 g, 9.0 mmol) in CH₂Cl₂ (100 mL). The mixture was stirred at that temperature for 1 hour and concentrated. The resulting crude product was purified by silica gel column chromatography to afford (R)-(15-bromo-1-butylpentadeca-2,11-diynyloxy)-tert-butyldimethylsilane (2.69 g, 5.73 mmol).

¹H-NMR (CDCl₃, 300 MHZ) δ ppm: 0.10 (s, 3H), 0.12 (s, 3H), 0.84-0.96 (m, 12H), 1.23-1.68 (m, 16H), 1.95-2.05 (m, 2H), 2.10-2.22 (m, 4H), 2.30-2.38 (m, 2H), 3.52 (t, J=6.5 Hz, 2H), 4.28-4.35 (m, 1H)

IR (neat): 2931, 2857, 2214, 1709, 1676, 1595, 1463, 1433, 1350, 1249, 1082, 1005, 938, 837, 778, 668, 566 cm⁻¹

(4) Aqueous hydrochloric acid (0.3 mL, 3.0M) was added at room temperature to a solution of the compound obtained in the above (3) (2.69 g, 5.73 mmol) in MeOH (50 mL), and the mixture was stirred at room temperature for 2.5 hours. To the reaction mixture was added a saturated aqueous NaHCO₃ (50 mL) and then the mixture was extracted with AcOEt (100 mL×2). The organic layer was washed with water (50 mL) and brine (50 mL), dried over anhydrous magnesium sulfate and concentrated. The resulting crude product was purified by silica gel column chromatography to afford (R)-19-bromononadeca-6,15-diyn-5-ol (1.51 g).

¹H-NMR (CDCl₃, 300 MHZ) δ ppm: 0.92 (t, J=7.1 Hz, 3H), 1.25-1.72 (m, 16H), 1.96-2.05 (m, 2H), 2.09-2.24 (m, 4H), 2.30-2.38 (m, 2H), 3.52 (t, J=6.5 Hz, 2H), 4.28-4.40 (m, 1H)

IR (neat): 3400, 2931, 2858, 2360, 1672, 1433, 1384, 1331, 1272, 1248, 1148, 1104, 1037 cm⁻¹

(5) A suspension of NaBH₄ (33 mg, 0.86 mmol) in EtOH (10 mL) was added dropwise, under a hydrogen atmosphere, to a solution of Ni(OAc)₂.4H₂O (122 mg, 0.43 mmol) in EtOH (10 mL) and the mixture was stirred at room temperature for 30 minutes. To the reaction mixture was added dropwise ethylenediamine (0.28 mL, 4.25 mmol) at room temperature, a solution of the compound obtained in the above (4) (1.51 g, 4.25 mmol) in EtOH (10 mL) was then added dropwise and the mixture was stirred at room temperature for about 3 hours until absorption of hydrogen gas ceased. To the reaction solution was added Et₂O (diethyl ether)(50 mL), the mixture was stirred for 10 minutes and then filtered through a silica gel pad and concentrated. The resulting crude product was purified by silica gel column chromatography to afford (R)-(6Z, 15Z)-19-bromononadeca-6,15-dien-5-ol (0.68 g).

¹H-NMR(CDCl₃, 300 MHz) δ ppm: 0.91 (t, J=6.8 Hz, 3H), 1.22-1.68 (m, 16H), 1.86-1.97 (m, 2H), 1.98-2.14 (m, 4H), 2.19 (q, J=7.4 Hz, 2H), 3.41 (t, J=6.7 Hz, 2H), 4.38-4.49 (m, 1H), 5.25-5.54 (m, 4H)

IR (neat): 3368, 3006, 2927, 2855, 2361, 1656, 1460, 1384, 1246, 1007, 727, 650, 565 cm⁻¹

(6) Sodium sulfite (517 mg, 4.1 mmol) and sodium iodide (205 mg, 1.364 mmol) were added at room temperature to a solution of the compound obtained in the above (5) (0.49 g, 1.364 mmol) in a mixed solvent of EtOH (20 mL) and water (20 mL), and the mixture was stirred under reflux for 4 hours. The reaction solution was concentrated and purified by silica gel column chromatography and resin (HP-20, Nippon Rensui) to afford the title compound (400 mg).

¹H-NMR (DMSO-d₆, 300 MHz) δ ppm: 0.85 (t, J=6.5 Hz, 3H), 1.13-1.67 (m, 18H), 1.89-2.10 (m, 6H), 2.33-2.41 (m, 2H), 4.12-4.28 (m, 1H), 4.44-4.51 (m, 1H), 5.20-5.42 (m, 4H)

IR (KBr): 3423, 3009, 2927, 2855, 2385, 2281, 1672, 1562, 1468, 1226, 1183, 1072, 797, 613, 427, 418 cm⁻¹

### Example 2

Sodium (R)-16-hydroxyeicosa-5,14-diyne-1-sulfonate (Compound No. 3)

(1) The reaction was carried out substantially in the same manner as Example 1 (1), but using 6-tetrahydropyranyloxy-1-hexyne instead of 5-tetrahydropyranyloxy-1-pentyne, followed by reaction in the same manner as Example 1 (2) to afford (R)-16-(tert-butyldimethylsilanyloxy)eicosa-5,14-diyn-1-ol.

¹H-NMR (CDCl₃, 300 MHz) δ ppm: 0.10 (s, 3H), 0.12 (s, 3H), 0.84-0.94 (m, 3H), 0.90 (s, 3H), 1.22-1.73 (m, 20H), 2.09-2.24 (m, 6H), 3.68 (t, J=6.3 Hz, 2H), 4.27-4.35 (m, 1H)

IR (neat): 3340, 2930, 2233, 1463, 1435, 1361, 1338, 1251, 1214, 1152, 1110, 1078, 1006, 983, 938, 899, 837, 777, 724, 668, 551 cm⁻¹

(2) Using the compound obtained in the above (1), the reaction was carried out in the same manner as Example 1 (3) to afford (R)-(16-bromo-1-butylhexadeca-2,11-diynyloxy)-tert-butyldimethylsilane.

¹H-NMR (CDCl₃, 300 MHz) δ ppm: 0.10 (s, 3H), 0.12 (s, 3H), 0.87-0.96 (m, 3H), 0.90 (s, 9H), 1.24-1.69 (m, 18H), 1.91-2.03 (m, 2H), 2.09-2.25 (m, 6H), 3.44 (t, J=6.8 Hz, 2H), 4.32 (tt, J=6.5, 2.0 Hz, 1H)

IR (neat): 3119, 2931, 2858, 2234, 1463, 1433, 1402, 1361, 1336, 1251, 1152, 1110, 1083, 1005, 938, 837, 778, 667, 564 cm⁻¹

(3) Using the compound obtained in the above (2), the reaction was carried out in the same manner as Example 1 (4) to afford (R)-20-bromoeicosa-6,15-diyn-5-ol.

¹H-NMR(CDCl₃, 300 MHz) δ ppm: 0.92 (t, J=7.1 Hz, 3H), 1.25-1.72 (m, 18H), 1.92-2.03 (m, 2H), 2.10-2.24 (m, 6H), 3.44 (t, J=6.8 Hz, 2H), 4.30-4.39 (m, 1H)

IR (neat): 3231, 2933, 2858, 2214, 1672, 1630, 1460, 1433, 1383, 1333, 1293, 1251, 1148, 1104, 1036, 730, 630, 596, 563 cm⁻¹

(4) Using the compound obtained in the above (3), the reaction was carried out in the same manner as Example 1 (6) to afford the title compound.

¹H-NMR (DMSO-d₆, 300 MHz) δ ppm: 0.86 (t, J=7.1 Hz, 3H), 1.18-1.68 (m, 20H), 2.04-2.21 (m, 6H), 2.33-2.43 (m, 2H), 4.09-4.19 (m, 1H), 5.08 (d, J=5.6 Hz, 1H)

IR (KBr): 3534, 2935, 2857, 2232, 1630, 1466, 1282, 1246, 1201, 1180, 1080, 1060, 892, 796, 728, 608, 536, 482, 421 cm⁻¹

### Example 3

Sodium (R)-(Z)-15-hydroxynonadec-13-ene-1-sulfonate (Compound No. 33)

(1) The reaction was carried out substantially in the same manner as Example 1 (1), but using 1,12-dibromododecane and (R)-3-tert-butyldimethylsilanyloxy-1-heptyne instead of 1,7-dibromoheptane and 5-tetrahydropyranyloxy-1-pentyne, respectively, to afford (R)-(15-bromo-1-butylpentadec-2-ynyloxy)-tert-butyldimethylsilane.

¹H-NMR (CDCl₃, 300 MHz) δ ppm: 0.10 (s, 3H), 0.12 (s, 3H), 0.88-0.92 (m, 12H), 1.24-1.52 (m, 22H), 1.58-1.67 (m, 2H), 1.80-1.93 (m, 2H), 2.18 (dt, J=2.0, 6.9 Hz, 2H), 3.41 (t, J=6.8 Hz, 2H), 4.31 (ddt, J=1.9, 1.9, 6.5 Hz, 1H)

IR (neat): 2930, 2856, 1464, 1361, 1341, 1251, 1152, 1110, 1083, 1005, 938, 838, 778, 667, 566 cm⁻¹

(2) Using the compound obtained in the above (1), the reaction was carried out in the same manner as Example 1 (4) to afford (R)-19-bromononadec-6-yn-5-ol.

¹H-NMR(CDCl₃, 300 MHz) δppm: 0.91 (t, J=7.1 Hz, 3H), 1.23-1.58 (m, 24H), 1.60-1.74 (m, 2H), 1.79-1.92 (m, 2H), 2.20 (dt, J=2.0, 7.0 Hz, 2H), 3.41 (t, J=6.8 Hz, 2H), 4.30-4.39 (m, 1H)

IR (neat): 3368, 2927, 2855, 2230, 1466, 1148, 1037, 722, 646, 563 cm⁻¹

(3) Using the compound obtained in the above (2), the reaction was carried out in the same manner as Example 1 (5) to afford (R)-(Z)-19-bromononadec-6-en-5-ol.

¹H-NMR(CDCl₃, 300 MHz) δ ppm: 0.91 (t, J=6.9 Hz, 3H), 1.20-1.65 (m, 24H), 1.79-1.92 (m, 2H), 2.01-2.15 (m, 2H), 3.41 (t, J=6.8 Hz, 2H), 4.37-4.47 (m, 1H), 5.31 (m, 2H)

IR (neat): 3368, 3005, 2925, 2854, 1656, 1466, 1378, 1251, 1008, 722, 647, 564 cm⁻¹

(4) Using the compound obtained in the above (3), the reaction was carried out in the same manner as Example 1 (6) to afford the title compound.

¹H-NMR (DMSO-d₆, 300 MHz) δ ppm: 0.90 (t, J=6.8 Hz, 3H), 1.20-1.61 (m, 26H), 1.90-2.07 (m, 2H), 2.31-2.41 (m, 2H), 4.13-4.25 (m, 1H), 4.46-4.53 (m, 1H), 5.21-5.53 (m, 2H)

IR (KBr): 3447, 3007, 2922, 2852, 1653, 1471, 1380, 1190, 1080, 1054, 968, 898, 798, 720, 611, 560, 535, 497, 471, 446, 418 cm⁻¹

### Example 4

Sodium (R)-15-hydroxynonadec-13-yne-1-sulfonate (Compound No. 10)

Using the compound obtained in Example 3 (2), the reaction was carried in the same manner as Example 1 (6) to afford the title compound.

¹H-NMR (DMSO-d₆, 300 MHz) δ ppm: 0.86 (t, J=7.0 Hz, 3H), 1.18-1.62 (m, 26H), 2.16 (dt, J=1.9, 6.6 Hz, 2H), 2.32-2.39 (m, 2H), 4.09-4.18 (m, 1H), 5.07 (d, J=5.4 Hz, 1H)

IR (KBr): 3366, 2920, 2851, 2229, 1656, 1472, 1380, 1195, 1181, 1064, 1011, 890, 799, 719, 613, 550, 530, 497, 432 cm⁻¹

### Example 5

Sodium (R)-(Z)-14-hydroxyoctadec-12-ene-1-sulfonate (Compound No. 42)

(1) The reaction was carried out substantially in the same manner as Example 1 (1), but using 1,11-dibromoundecane and (R)-3-tert-butyldimethylsilanyloxy-1-heptyne instead of 1,7-dibromoheptane and 5-tetrahydropyranyloxy-1-pentyne, respectively, to afford (R)-(14-bromo-1-butyltetradec-2-ynyloxy)-tert-butyldimethylsilane.

¹H-NMR (CDCl₃, 300 MHz) δ ppm: 0.10 (s, 3H), 0.12 (s, 3H), 0.84-0.96 (m, 12H), 1.20-1.68 (m, 26H), 1.80-1.91 (m, 2H), 2.18 (dt, J=1.9, 6.9 Hz, 2H), 3.41 (t, J=6.8 Hz, 2H), 4.27-4.35 (m, 1H)

IR (neat): 2929, 2856, 1464, 1361, 1341, 1251, 1110, 1083, 1006, 938, 837, 778, 667, 565 cm⁻¹

(2) Using the compound obtained in the above (1), the reaction was carried out in the same manner as Example 1 (4) to afford (R)-18-bromooctadec-6-yn-5-ol.

¹H-NMR(CDCl₃, 300 MHz) δ ppm: 0.92 (t, J=7.1 Hz, 3H), 1.21-1.57 (m, 20H), 1.60-1.74 (m, 2H), 1.80-1.92 (m, 2H), 2.20 (dt, J=2.0, 7.0 Hz, 1H), 3.41 (t, J=6.9 Hz, 2H), 4.30-4.40 (m, 1H)

IR (neat): 3368, 2929, 2855, 2215, 1672, 1466, 1384, 1148, 1039, 723, 646, 564 cm⁻¹

(3) Using the compound obtained in the above (2), the reaction was carried out in the same manner as Example 1 (5) to afford (R)-(Z)-18-bromooctadec-6-en-5-ol.

¹H-NMR (CDCl₃, 300 MHZ) δ ppm: 0.91 (t, J=6.9 Hz, 3H), 1.18-1.67 (m, 22H), 1.70-1.82 (m, 2H), 1.97-2.18 (m, 2H), 3.53 (t, J=6.8 Hz, 2H), 4.37-4.48 (m, 1H), 5.30-5.41 (m, 1H), 5.43-5.54 (m, 1H)

IR (neat): 3368, 2927, 2855, 1466, 1379, 1311, 1007, 729, 654 cm⁻¹

(4) Using the compound obtained in the above (3), the reaction was carried out in the same manner as Example 1 (6) to afford the title compound.

¹H-NMR (DMSO-d₆, 300 MHz) δ ppm: 0.85 (t, J=6.7 Hz, 3H), 1.12-1.59 (m, 24H), 1.92-2.05 (m, 2H), 2.31-2.39 (m, 2H), 4.16-4.26 (m, 1H), 4.46 (d, J=4.7 Hz, 1H), 5.21-5.53 (m, 2H)

IR (KBr): 3359, 2923, 2852, 1656, 1468, 1379, 1185, 1055, 1024, 970, 898, 797, 722, 610, 557, 531, 420 cm⁻¹

### Example 6

Sodium (R)-14-hydroxynonadec-12-yne-1-sulfonate (Compound No. 7)

(1) The reaction was carried out substantially in the same manner as Example 1 (1), but using 1,11-dibromoundecane and (R)-3-tert-butyldimethylsilanyloxy-1-octyne instead of 1,7-dibromoheptane and 5-tetrahydropyranyloxy-1-pentyne, respectively, followed by the reaction in the same manner as Example 1 (4) to afford (R)-19-bromononadec-7-yn-6-ol.

¹H-NMR(CDCl₃, 300 MHz) δ ppm: 0.90 (t, J=7.0 Hz, 3H), 1.24-1.56 (m, 22H), 1.60-1.74 (m, 2H), 1.80-1.91 (m, 2H), 2.20 (dt, J=2.0, 7.0 Hz, 2H), 3.41 (t, J=6.9 Hz, 2H), 4.30-4.39 (m, 1H)

IR (neat): 3400, 2928, 2855, 2212, 1672, 1466, 1384, 1148, 1024, 723, 646, 564 cm⁻¹

(2) Using the compound obtained in the above (1), the reaction was carried in the same manner as Example 1 (6) to afford the title compound.

¹H-NMR (DMSO-d₆, 300 MHz) δ ppm: 0.86 (t, J=6.8 Hz, 3H), 1.16-1.70 (m, 26H), 2.11-2.20 (m, 2H), 2.32-2.40 (m, 2H), 4.09-4.19 (m, 1H), 5.07 (d, J=5.4 Hz, 1H)

IR (KBr): 3509, 2919, 2850, 2229, 1659, 1466, 1412, 1304, 1277, 1228, 1212, 1161, 1085, 1062, 914, 799, 723, 622, 548, 535, 420 cm⁻¹

### Example 7

Sodium (R)-(Z)-14-hydroxynonadec-12-ene-1-sulfonate (Compound No. 29)

(1) Using the compound obtained in Example 6 (1), the reaction was carried out in the same manner as Example 1 (5) to afford (R)-(Z)-19-bromononadec-7-en-6-ol.

¹H-NMR (CDCl₃, 300 MHz) δ ppm: 0.89 (t, J=6.7 Hz, 3H), 1.20-1.67 (m, 24H), 1.79-1.91 (m, 2H), 1.98-2.16 (m, 2H), 3.41 (t, J=6.9 Hz, 2H), 4.37-4.47 (m, 1H), 5.32-5.40 (m, 1H), 5.43-5.53 (m, 1H)

IR (neat): 3368, 3005, 2926, 2854, 1658, 1466, 1384, 1255, 1123, 1084, 1022, 724, 647, 564 cm⁻¹

(2) Using the compound obtained in the above (1), the reaction was carried out in the same manner as Example 1 (6) to afford the title compound.

¹H-NMR (DMSO-d₆, 300 MHz) δ ppm: 0.85 (t, J=6.7 Hz, 3H), 1.16-1.59 (m, 26H), 1.92-2.06 (m, 2H), 2.30-2.39 (m, 2H), 4.15-4.25 (m, 1H), 4.46-4.50 (m, 1H), 5.20-5.39 (m, 2H)

IR (KBr): 3358, 2921, 2852, 1656, 1469, 1411, 1379, 1207, 1191, 1084, 1051, 910, 796, 722, 608, 542, 530, 446, 420 cm⁻¹

### Example 8

Sodium (R)-(Z)-16-hydroxyeicos-14-ene-1-sulfonate (Compound No. 30)

(1) The reaction was carried out substantially in the same manner as Example 1 (1), but-using 1,13-dibromotridecane and (R)-3-tert-butyldimethylsilanyloxy-1-heptyne instead of 1,7-dibromoheptane and 5-tetrahydropyranyloxy-1-pentyne, respectively, followed by the reactions in the same manner as Example 1 (4) and Example 1 (5) to afford (R)-(Z)-20-bromoeicos-6-en-5-ol.

¹H-NMR(CDCl₃, 300 MHz) δ ppm: 0.90 (t, J=6.8 Hz, 3H), 1.19-1.64 (m, 26H), 1.79-1.92 (m, 2H), 1.97-2.17 (m, 2H), 3.41 (t, J=6.8 Hz, 2H), 4.38-4.47 (m, 1H), 5.31-5.41 (m, 1H), 5.42-5.54 (m, 1H)

IR (neat): 3152, 3006, 2925, 2854, 1466, 1401, 1008, 723, 647, 564 cm⁻¹

(2) Using the compound obtained in the above (1), the reaction was carried out in the same manner as Example 1 (6) to afford the title compound.

¹H-NMR (DMSO-d₆, 300 MHz) δ ppm: 0.85 (t, J=6.6 Hz, 3H), 1.15-1.59 (m, 28H), 1.91-2.06 (m, 2H), 2.30-2.40 (m, 2H), 4.13-4.25 (m, 1H), 4.48 (d, J=4.5 Hz, 1H), 5.20-5.40 (m, 2H)

IR (KBr): 3508, 3360, 3008, 2919, 2850, 1660, 1468, 1410, 1221, 1161, 1060, 964, 898, 799, 722, 623, 547, 534, 450, 418 cm⁻¹

### Example 9

Sodium (S)-(Z)-15-hydroxynonadec-13-ene-1-sulfonate (Compound No. 34)

(1) The reaction was carried out substantially in the same manner as Example 1 (1), but using 1,12-dibromododecane and (S)-3-tert-butyldimethylsilanyloxy-1-heptyne instead of 1,7-dibromoheptane and 5-tetrahydropyranyloxy-1-pentyne, respectively, followed by the reaction in the same manner as Example 1 (4) to afford (S)-19-bromononadec-6-yn-5-ol.

¹H-NMR(CDCl₃, 300 MHZ) δ ppm: 0.92 (t, J=7.1 Hz, 3H), 1.20-1.75 (m, 24H), 1.80-1.92 (m, 2H), 2.20 (dt, J=1.9, 7.0 Hz, 2H), 3.41 (t, J=6.9 Hz, 2H), 4.29-4.40 (m, 1H)

IR (neat): 3229, 2927, 2854, 1630, 1461, 1404, 1384, 1294, 1148, 1036, 722, 629, 596 cm⁻¹

(2) Using the compound obtained in the above (1), the reaction was carried out in the same manner as Example 1 (5) to afford (S)-(Z)-19-bromononadec-6-en-5-ol.

¹H-NMR (CDCl₃, 300 MHz) δ ppm: 0.91 (t, J=6.8 Hz, 3H), 1.20-1.66 (m, 24H), 1.79-1.91 (m, 2H), 1.98-2.15 (m, 2H), 3.41 (t, J=6.8 Hz, 2H), 4.37-4.47 (m, 1H), 5.31-5.40 (m, 1H), 5.43-5.54 (m, 1H)

IR (neat): 3118, 3010, 2926, 2854, 1466, 1401, 1084, 1021, 723, 648, 564, 500 cm⁻¹

(3) Using the compound obtained in the above (2), the reaction was carried out in the same manner as Example 1 (6) to afford the title compound.

¹H-NMR (DMSO-d₆, 300 MHz) δ ppm: 0.85 (t, J=6.6 Hz, 3H), 1.12-1.58 (m, 26H), 1.92-2.05 (m, 2H), 2.30-2.38 (m, 2H), 4.13-4.25 (m, 1H), 4.47 (d, J=4.5 Hz, 1H), 5.21-5.35 (m, 2H)

IR (KBr): 3445, 2921, 2852, 1656, 1470, 1379, 1190, 1054, 798, 720, 613, 560, 535, 424, 418 cm⁻¹

### Example 10

Sodium (RS)-17-hydroxyhenicos-15-yne-1-sulfonate (Compound No. 9)

(1) The reaction was carried out substantially in the same manner as Example 1 (1), but using 1,14-dibromotetradecane and (RS)-3-tert-butyldimethylsilanyloxy-1-heptyne instead of 1,7-dibromoheptane and 5-tetrahydropyranyloxy-1-pentyne, respectively, followed by the reaction in the same manner as Example 1 (4) to afford (RS)-21-bromohenicos-6-yn-5-ol.

¹H-NMR(CDCl₃, 300 MHz) δ ppm: 0.92 (t, J=7.1 Hz, 3H), 1.19-1.74 (m, 28H), 1.79-1.92 (m, 2H), 2.20 (dt, J=2.0, 7.0 Hz, 2H), 3.41 (t, J=6.8 Hz, 2H), 4.30-4.40 (m, 1H)

IR (neat): 3232, 2926, 2854, 2215, 1630, 1466, 1384, 1294, 1148, 1036, 723, 645, 596 cm⁻¹

(2) Using the compound obtained in the above (1), the reaction was carried out in the same manner as Example 1 (6) to afford the title compound.

¹H-NMR (DMSO-d₆, 300 MHz) δ ppm: 0.86 (t, J=7.1 Hz, 3H), 1.10-1.60 (m, 30H), 2.12-2.20 (m, 2H), 2.32-2.40 (m, 2H), 4.09-4.19 (m, 1H), 5.07 (d, J=5.6 Hz, 1H)

IR (KBr): 3508, 2920, 2850% 2226, 1661, 1470, 1410, 1380, 1300, 1254, 1234, 1220, 1160, 1060, 960, 890, 799, 721, 623, 548, 534, 434 cm⁻¹

### Example 11

Sodium (R)-10-hydroxytetradec-8-yne-1-sulfonate (Compound No. 11)

(1) The reaction was carried out substantially in the same manner as Example 1 (1), but using (R)-3-tert-butyldimethylsilanyloxy-1-heptyne instead of 5-tetrahydropyranyloxy-1-pentyne, to afford (R)-(10-bromo-1-butyldec-2-ynyloxy)-tert-butyldimethylsilane.

¹H-NMR (CDCl₃, 300 MHz) δ ppm: 0.10 (s, 3H), 0.12 (s, 3H), 0.84-0.96 (m, 3H), 0.91 (s, 9H), 1.24-1.68 (m, 14H), 1.80-1.92 (m, 2H), 2.19 (dt, J=1.9, 6.9 Hz, 2H), 3.41 (t, J=6.4 Hz, 2H), 4.32 (tt, J=6.5, 1.9 Hz, 1H)

IR (neat): 2930, 2858, 2233, 1463, 1407, 1389, 1361, 1341, 1251, 1217, 1152, 1110, 1083, 1006, 938, 837, 778, 725, 667, 565 cm⁻¹

(2) Using the compound obtained in the above (1), the reaction was carried in the same manner as Example 1 (4) to afford (R)-14-bromotetradec-6-yn-5-ol.

¹H-NMR(CDCl₃, 300 MHz) δ ppm: 0.92 (t, J=7.1 Hz, 3H), 1.24-1.75 (m, 14H), 1.80-1.92 (m, 2H), 2.21 (dt, J=2.0, 6.9 Hz, 2H), 3.41 (t, J=6.8 Hz, 2H), 4.31-4.39 (m, 1H)

IR (neat): 3231, 2932, 2858, 1630, 1461, 1384, 1294, 1148, 1104, 1036, 726, 630, 596, 563, 418 cm⁻¹

(3) Using the compound obtained in the above (2), the reaction was carried out in the same manner as Example 1 (6) to afford the title compound.

¹H-NMR (DMSO-d₆, 300 MHz) δ ppm: 0.86 (t, J=7.1 Hz, 3H), 1.18-1.60 (m, 16H), 2.16 (dt, J=1.9, 6.8 Hz, 2H), 2.32-2.40 (m, 2H), 4.09-4.19 (m, 1H), 5.08 (d, J=5.6 Hz, 1H)

IR (KBr): 3324, 2934, 2858, 2230, 1648, 1467, 1332, 1234, 1186, 1059, 1011, 890, 798, 727, 612, 547, 529, 418 cm⁻¹

### Example 12

Sodium (RS)-15-hydroxy-15-methyleicos-13-yne-1-sulfonate (Compound No. 8)

(1) The reaction was carried out substantially in the same manner as Example 1 (1), but using 1,12-dibromododecane and (RS)-3-triethylsilanyloxy-3-methyl-1-octyne instead of 1,7-dibromoheptane and 5-tetrahydropyranyloxy-1-pentyne, respectively, followed by the reaction in the same manner as Example 1 (4) to afford (RS)-20-bromo-6-methyleicos-7-yn-6-ol.

¹H-NMR(CDCl₃, 300 MHz) δ ppm: 0.90 (d, J=6.9 Hz, 3H), 1.20-1.68 (m, 29H), 1.74-1.91 (m, 2H), 2.18 (t, J=7.0 Hz, 2H), 3.41 (t, J=6.8 Hz, 2H)

IR (neat): 3119, 2929, 2855, 2238, 1465, 1399, 1128, 1056, 934, 772, 724, 647, 563 cm⁻¹

(2) Using the compound obtained in the above (1), the reaction was carried out in the same manner as Example 1 (6) to afford the title compound.

¹H-NMR (DMSO-d₆, 300 MHz) δ ppm: 0.86 (t, J=6.9 Hz, 3H), 1.15-1.59 (m, 31H), 2.14 (t, J=6.5 Hz, 2H), 2.30-2.40 (m, 2H), 4.96 (s, 1H)

IR (KBr): 3529, 2920, 2850, 2236, 1660, 1470, 1409, 1376, 1268, 1244, 1225, 1161, 1058, 943, 895, 799, 721, 623, 547, 533, 490, 418 cm⁻¹

### Example 13

Sodium (RS)-15-hydroxy-17-methyloctadec-13-yne-1-sulfonate (Compound No. 12)

(1) The reaction was carried out substantially in the same manner as Example 1 (1), but using 1,12-dibromododecane and (RS)-3-tert-butyldimethylsilanyloxy-5-methyl -1-hexyne instead of 1,7-dibromoheptane and 5-tetrahydropyranyloxy-1-pentyne, respectively, followed by the reaction in the same manner as Example 1 (4) to afford (RS)-18-bromo-2-methyloctadec-5-yn-4-ol.

¹H-NMR (CDCl₃, 300 MHz) δ ppm: 0.89-0.97 (m, 6H), 1.20-1.67 (m, 20H), 1.76-1.92 (m, 3H), 2.20 (dt, J=2.0, 7.0 Hz, 2H), 3.41 (t, J=6.8 Hz, 2H), 4.35-4.45 (m, 1H)

IR (neat): 3228, 2927, 2854, 1630, 1466, 1404, 1385, 1367, 1294, 1153, 1036, 722, 629, 596 cm⁻¹

(2) Using the compound obtained in the above (1), the reaction was carried out in the same manner as Example 1 (6) to afford the title compound.

¹H-NMR (DMSO-d₆, 300 MHz) δ ppm: 0.85 (d, J=6.5 Hz, 3H), 0.87 (d, J=6.7 Hz, 3H), 1.16-1.60 (m, 22H), 1.66-1.82 (m, 1H), 2.16 (dt, J=1.9, 6.7 Hz, 2H), 2.32-2.39 (m, 2H), 4.13-4.23 (m, 1H), 5.05 (d, J=5.8 Hz, 1H)

IR (KBr): 3540, 2918, 2852, 2235, 1638, 1472, 1369, 1297, 1268, 1204, 1186, 1119, 1056, 966, 837, 801, 719, 611, 536, 481 cm⁻¹

### Example 14

Sodium (S)-15-cyclohexyl-15-hydroxypentadec-13-yne-1-sulfonate (Compound No. 13)

(1) The reaction was carried out substantially in the same manner as Example 1 (1), but using 1,12-dibromododecane and (S)-3-tert-butyldimethylsilanyloxy-3-cyclohexyl -1-propyne instead of 1,7-dibromoheptane and 5-tetrahydropyranyloxy-1-pentyne, respectively, followed by the reaction in the same manner as Example 1 (4) to afford (S)-15-bromo-1-cyclohexylpentadec-2-yn-1-ol.

¹H-NMR (CDCl₃, 300 MHz) δ ppm: 0.98-1.91 (m, 31H), 2.21 (dt, J=2.0, 7.0 Hz, 2H), 3.41 (t, J=6.8 Hz, 2H), 4.10-4.17 (m, 1H)

IR (neat): 3119, 2925, 2853, 1450, 1399, 1084, 1010, 893, 722, 647, 563 cm⁻¹

(2) Using the compound obtained in the above (1), the reaction was carried out in the same manner as Example 1 (6) to afford the title compound.

¹H-NMR (DMSO-d₆, 300 MHz) δ ppm: 0.87-1.82 (m, 31H), 2.12-2.21 (m, 2H), 2.31-2.40 (m, 2H), 3.90-3.97 (m, 1H), 5.01 (d, J=5.6 Hz, 1H)

IR (KBr): 3396, 2920, 2851, 2235, 1627, 1472, 1454, 1272, 1179, 1055, 1005, 890, 799, 782, 752, 718, 676, 609, 552, 528, 497, 426 cm⁻¹

### Example 15

Sodium (S)-15-hydroxy-16-phenylhexadec-13-yne-1-sulfonate (Compound No. 15)

(1) The reaction was carried out substantially in the same manner as Example 1 (1), but using 1,12-dibromododecane and (S)-3-tert-butyldimethylsilanyloxy-4-phenyl -1-butyne instead of 1,7-dibromoheptane and 5-tetrahydropyranyloxy-1-pentyne, respectively, followed by the reaction in the same manner as Example 1 (4) to afford (S)-16-bromo-1-phenylhexadec-3-yn-2-ol.

¹H-NMR (CDCl₃, 300 MHz) δ ppm: 1.21-1.58 (m, 18H), 1.80-1.91 (m, 2H), 2.19 (dt, J=2.0, 7.0 Hz, 2H), 2.95 (dd, J=13.4, 6.8 Hz, 1H), 3.01 (dd, J=13.4, 6.3 Hz, 1H), 3.41 (t, J=6.8 Hz, 2H), 4.52-4.62 (m, 1H), 7.21-7.35 (m, 5H)

IR (neat): 3229, 3001, 2924, 2853, 1630, 1495, 1455, 1404, 1385, 1294, 1036, 739, 699, 629, 596 cm⁻¹

(2) Using the compound obtained in the above (1), the reaction was carried out in the same manner as Example 1 (6) to afford the title compound.

¹H-NMR (DMSO-d₆, 300 MHz) δ ppm: 0.98-1.62 (m, 20H), 2.12 (dt, J=1.8, 6.7 Hz, 2H), 2.32-2.40 (m, 2H), 2.76 (dd, J=13.1, 6.9 Hz, 1H), 2.85 (dd, J=13.1, 6.8 Hz, 1H), 4.29-4.39 (m, 1H), 5.31 (d, J=5.8 Hz, 1H), 7.41-7.29 (m, 5H)

IR (KBr): 3384, 3030, 2919, 2850, 2227, 1659, 1497, 1471, 1455, 1426, 1224, 1160, 1057, 846, 798, 742, 720, 698, 621, 545, 473 cm⁻¹

### Example 16

Sodium (R)-15-hydroxy-16-phenoxyhexadec-13-yne-1-sulfonate (Compound No. 17)

(1) The reaction was carried out substantially in the same manner as Example 1 (1), but using 1,12-dibromododecane and (R)-3-tert-butyldimethylsilanyloxy-4-phenoxy -1-butyne instead of 1,7-dibromoheptane and 5-tetrahydropyranyloxy-1-pentyne, respectively, followed by the reaction in the same manner as Example 1 (4) to afford (R)-16-bromo-1-phenoxyhexadec-3-yn-2-ol.

¹H-NMR (CDCl₃, 300 MHz) δ ppm: 1.23-1.58 (m, 18H), 1.78-1.91 (m, 2H), 2.23 (dt, J=2.0, 7.1 Hz, 2H), 2.33-2.42 (m, 1H), 3.40 (t, J=6.8 Hz, 2H), 4.02 (dd, J=9.6, 7.7 Hz, 1H), 4.11 (dd, J=9.6, 3.6 Hz, 1H), 4.71-4.80 (m, 1H), 6.90-7.02 (m, 3H), 7.25-7.34 (m, 2H)

IR (neat): 3400, 2927, 2854, 2238, 1600, 1588, 1497, 1456, 1401, 1301, 1246, 1173, 1143, 1081, 1045, 903, 754, 691, 645, 562, 509 cm⁻¹

(2) Using the compound obtained in the above (1), the reaction was carried out in the same manner as Example 1 (6) to afford the title compound.

¹H-NMR (DMSO-d₆, 300 MHz) δ ppm: 1.14-1.60 (m, 20H), 2.19 (dt, J=1.8, 6.8 Hz, 2H), 2.31-2.39 (m, 2H), 3.88-3.99 (m, 2H), 4.48-4.57 (m, 1H), 5.59 (d, J=5.9 Hz, 1H), 6.89-6.97 (m, 3H), 7.23-7.32 (m, 2H)

IR (KBr): 3412, 2920, 2850, 1602, 1588, 1501, 1471, 1451, 1306, 1256, 1212, 1183, 1070, 1044, 896, 853, 788, 753, 721, 694, 620, 546 cm⁻¹

### Example 17

Sodium 14-(1-hydroxycyclopentyl)tetradec-13-yne-1-sulfonate (Compound No. 18)

(1) The reaction was carried out substantially in the same manner as Example 1 (1), but using 1,12-dibromododecane and 1-ethynyl-1-triethylsilanyloxycyclopentane instead of 1,7-dibromoheptane and 5-tetrahydropyranyloxy -1-pentyne, respectively, followed by the reaction in the same manner as Example 1 (4) to afford 1-(14-bromotetradec-1-ynyl)cyclopentanol.

¹H-NMR (CDCl₃, 300 MHz) δ ppm: 1.19-2.00 (m, 28H), 2.19 (t, J=7.1 Hz, 2H), 3.41 (t, J=6.8 Hz, 2H)

IR (neat): 3228, 2927, 2854, 2360, 1630, 1461, 1404, 1385, 1294, 1219, 1063, 1036, 994, 723, 629, 596, 564 cm⁻¹

(2) Using the compound obtained in the above (1), the reaction was carried out in the same manner as Example 1 (6) to afford the title compound.

¹H-NMR (DMSO-d₆, 300 MHz) δ ppm: 1.15-1.82 (m, 28H), 2.15 (t, J=6.8 Hz, 2H), 2.31-2.39 (m, 2H), 4.96 (s, 1H)

IR (KBr): 3530, 2920, 2850, 1656, 1627, 1471, 1356, 1224, 1165, 1082, 1057, 993, 879, 800, 722, 613, 554, 528, 485, 426 cm⁻¹

### Example 18 Sodium (R)-15-hydroxynonadecane-1-sulfonate (Compound No. 53)

A suspension of Pd (5 mg, 5 wt % on activated carbon) and the compound (100 mg, 0.26 mmol) obtained in Example 3 in MeOH (5 mL) was stirred at room temperature for about 4 hours until absorption of hydrogen gas ceased. The mixture was filtered through a celite pad and concentrated to afford the title compound (87 mg).

¹H-NMR (DMSO-d₆, 300 MHz) δ ppm: 0.86 (t, J=6.8 Hz, 3H), 1.15-1.61 (m, 32H), 2.31-2.39 (m, 2H), 3.27-3.39 (m, 1H), 4.19 (d, J=5.3 Hz, 1H)

IR (KBr): 3330, 2919, 2851, 1708, 1469, 1418, 1379, 1346, 1183, 1133, 1069, 1058, 937, 878, 857, 798, 722, 622, 536, 420 cm⁻¹

### Example 19

Sodium (R)-(Z)-15-acetoxynonadec-13-ene-1-sulfonate (Compound No. 31)

(1) Acetic anhydride (657 mg, 6.44 mmol) was added at 0° C. to a solution of the compound obtained in Example 3 (3) (1.55 g, 4.29 mmol), DMAP ((4-dimethylamino)pyridine) (10 mg, 0.082 mmol) and pyridine (678 mg, 8.58 mmol) in THF (45 mL), and the mixture was stirred at room temperature overnight. The reaction mixture was poured into water and then the mixture was extracted with AcOEt (100 mL×2). The organic layer was washed with aqueous hydrochloric acid (5 mL, 3.0M) and brine, dried over anhydrous magnesium sulfate and concentrated. The resulting crude product was purified by silica gel column chromatography to afford (R)-(Z)-5-acetoxy-19-bromononadec-6-ene (1.60 g).

¹H-NMR(CDCl₃, 300 MHZ) δ ppm: 0.89 (t, J=6.9 Hz, 3H), 1.18-1.73 (m, 24H), 1.80-1.91 (m, 2H), 2.02 (s, 3H), 2.05-2.21 (m, 2H), 3.41 (t, J=6.9 Hz, 2H), 5.24-5.33 (m, 1H), 5.47-5.58 (m, 2H)

IR (neat): 3468, 2927, 2855, 2360, 1737, 1466, 1370, 1241, 1018, 955, 723, 648, 608, 564 cm⁻¹

(2) Using the compound obtained in the above (1), the reaction was carried out in the same manner as Example 1 (6) to afford the title compound.

¹H-NMR (DMSO-d₆, 300 MHz) δ ppm: 0.85 (t, J=7.0 Hz, 3H), 1.14-1.68 (m, 26H), 1.97 (s, 3H), 2.01-2.12 (m, 2H), 2.31-2.40 (m, 2H), 5.24-5.34 (m, 1H), 5.39-5.56 (m, 2H)

IR (KBr): 3630, 3549, 2920, 2853, 1740, 1624, 1469, 1372, 1245, 1200, 1180, 1055, 1019, 958, 865, 796, 722, 609, 535, 482, 417 cm⁻¹

### Example 20

Sodium (S)-(E)-15-hydroxynonadec-13-ene-1-sulfonate (Compound No. 44)

(1) n-BuLi (46.8 mL, 2.66M in hexane, 124.4 mmol) was added dropwise at −60° C. over 15 minutes, under argon stream, to a solution of 12-bromo-1-dodecanol (15.0 g, 56.6 mmol) and (R)-3-tert-butyldimethylsilanyloxy-1-heptyne (10.67 g, 47.1 mmol) in a mixed solvent of THF (200 mL) and DMPU (100 mL). Thereafter, the temperature of the reaction solution was allowed to rise up to 0° C. over 45 minutes. To the resulting solution was added aqueous hydrochloric acid (100 mL, 3.0M) and the mixture was extracted with AcOEt (150 mL×2). The organic layer was washed with brine (200 mL), dried over anhydrous magnesium sulfate and concentrated. The resulting crude product was purified by silica gel column chromatography to afford (R)-15-(tert-butyldimethylsilanyloxy)nonadec-13-yn-1-o 1 (18.0 g).

¹H-NMR (CDCl₃, 300 MHz) δ ppm: 0.10 (s, 3H), 0.12 (s, 3H), 0.85-0.96 (m, 12H), 1.15-1.70 (m, 26H), 2.18 (dt, J=1.9, 6.9 Hz, 2H), 3.64 (m, J=6.6 Hz, 2H), 4.11 (tt, J=6.5, 1.9 Hz, 1H)

IR (neat): 3368, 2929, 2855, 2361, 1463, 1385, 1250, 1079, 938, 837, 777 cm⁻¹

(2) Using the compound obtained in the above (1), the reaction was carried out in the same manner as Example 1 (4) to afford (R)-nonadec-13-yne-1,15-diol.

¹H-NMR(CDCl₃, 300 MHz) δ ppm: 0.92 (t, J=7.1 Hz, 3H), 1.21-1.74 (m, 26H), 2.20 (dt, J=1.9, 7.0 Hz, 2H), 3.64 (m, J=6.6 Hz, 2H), 4.35 (tt, J=6.5, 1.9 HZ, 1H)

IR (KBr): 3197, 2919, 2853, 1741, 1466, 1324, 1277, 1144, 1112, 1053, 1015, 992, 968, 895, 812, 724, 643, 545, 494, 452 cm⁻¹

(3) Diethyl azodicarboxylate (335 mg, 40% in toluene solution, 1.92 mmol) was added at 0° C. to a solution of the compound obtained above (2) (190 mg, 0.64 mmol), benzoic acid (235 mg, 1.92 mmol) and triphenylphosphine (504 mg, 1.92 mmol) in THF (20 mL), and the mixture was stirred at that temperature for 30 minutes. The reaction mixture was concentrated and purified by silica gel column chromatography to afford benzoic acid (S)-15-benzoyloxynonadec-13-ynyl ester. To a solution of that compound in MeOH (10 mL) was added sodium methoxide (139 mg, 2.56 mmol) at room temperature, and the mixture was stirred at that temperature for 1.5 hours. To the resulting solution was added aqueous hydrochloric acid (10 mL, 3.0M) and extracted with AcOEt (20 mL×2). The organic layer was washed with brine (30 mL), dried over anhydrous magnesium sulfate and concentrated. The resulting crude product was purified by silica gel column chromatography to afford (S)-nonadec-13-yne-1,15-diol (170 mg).

¹H-NMR(CDCl₃, 300 MHz) δ ppm: 0.92 (t, J=7-1 Hz, 3H), 1.19-1.77 (m, 26H), 2.20 (dt, J=1.9, 7.0 Hz, 2H), 3.64 (t, J=6.6 Hz, 2H), 4.35 (tt, J=6.6, 1.9 Hz, 1H)

IR (KBr): 3314, 2919, 2852, 1741, 1465, 1324, 1276, 1193, 1144, 1112, 1069, 1015, 992, 968, 895, 803, 724, 622, 545, 494 cm⁻¹

(4) Lithium aluminum hydride (41 mg, 1.08 mmol) was added at room temperature to a solution of sodium methoxide (117 mg, 2.16 mmol) in THF (20 mL) under argon stream. To the mixture was added the compound obtained in the above (3) (160 mg, 0.54 mmol) and then the mixture was stirred at 70° C. for 1.5 hours. To the resulting solution was added water and aqueous hydrochloric acid (5.0 mL, 3.0M) and the mixture was extracted with AcoEt (50 mL). The organic layer was washed with brine (50 mL), dried over anhydrous magnesium sulfate and concentrated. The resulting crude product was purified by silica gel column chromatography to afford (S)-(E)-nonadec-13-ene-1,15-diol (119 mg).

¹H-NMR(CDCl₃, 300 MHZ) δ ppm: 0.90 (t, J=6.8 Hz, 3H), 1.20-1.63 (m, 26H), 1.97-2.07 (m, 2H), 3.64 (t, J=6.6 Hz, 2H), 4.03 (q, J=6.6 Hz, 1H), 5.40-5.50 (m, 1H), 5.57-5.69 (m, 1H)

IR (KBr): 3267, 2956, 2917, 2851, 1672, 1471, 1380, 1341, 1146, 1126, 1058, 1012, 981, 958, 884, 788, 720, 527, 499, 460 cm⁻¹

(5) Triethylamine (50 μL, 0.38 mmol) was added at 0° C., under argon stream, to a solution of the compound obtained in the above (4) (160 mg, 0.54 mmol) in CH₂Cl₂ (20 mL). To the mixture was added dropwise methanesulfonyl chloride (30 μL, 0.38 mmol) at room temperature, and the mixture was stirred at that temperature for 1.5 hours. To the reaction mixture was added water and aqueous hydrochloric acid (5 mL, 3.0M) and then the mixture was extracted with Et₂O (50 mL). The organic layer was washed with water (50 mL) and brine (50 mL), dried over anhydrous magnesium sulfate and concentrated. To a solution of the resulting crude product in acetone (20 mL) was added lithium bromide (120 mg, 1.34 mmol) and then the mixture was stirred under reflux for 5 hours. To the reaction mixture was added water and then the mixture was extracted with AcOEt (50 mL×2). The organic layer was washed with brine (100 mL), dried over anhydrous magnesium sulfate and concentrated. The resulting crude product was purified by column chromatography to afford (S)-(E)-19-bromononadec-6-en-5-ol (70 mg).

¹H-NMR (CDCl₃, 300 MHZ) δ ppm: 0.90 (t, J=6.8 Hz, 3H), 1.18-1.62 (m, 24H), 1.80-1.91 (m, 2H), 1.97-2.07 (m, 2H), 3.41 (t, J=6.8 Hz, 2H), 3.99-4.09 (m, 1H), 5.40-5.50 (m, 1H), 5.58-5.69 (m, 1H)

IR (neat): 3368, 2924, 2854, 1670, 1466, 1378, 1262, 1126, 1006, 969, 898, 723, 647, 564 cm⁻¹

(6) Using the compound obtained in the above (5), the reaction was carried out in the same manner as Example 1 (6) to afford the title compound.

¹H-NMR (DMSO-d₆, 300 MHz) δ ppm: 0.86 (t, J=6.6 Hz, 3H), 1.24-1.59 (m, 26H), 1.91-2.01 (m, 2H), 2.31-2.39 (m, 2H), 3.78-3.88 (m, 1H), 4.49 (d, J=4.7 Hz, 1H), 5.30-5.40 (m, 1H), 5.43-5.54 (m, 1H)

IR (KBr): 3540, 3486, 2919, 2852, 1636, 1472, 1202, 1179, 1056, 967, 899, 801, 720, 611, 536, 483, 429 cm⁻¹

### Example 21

Sodium (R)-(E)-15-hydroxynonadec-13-ene-1-sulfonate (Compound No. 43)

(1) The reaction was carried out substantially in the same manner as Example 20 (4), but using the compound obtained in Example 20 (2) instead of (S)-nonadec-13-yne -1,15-diol, to afford (R)-(E)-nonadec-13-ene-1,15-diol.

¹H-NMR(CDCl₃, 300 MHz) δ ppm: 0.90 (t, J=6.9 Hz, 3H), 1.22-1.74 (m, 26H), 1.97-2.07 (m, 2H), 3.64 (t, J=6.6 Hz, 2H), 3.99-4.07 (m, 1H), 5.40-5.50 (m, 1H), 5.57-5.69 (m,

IR (neat): 3340, 2925, 2854, 1711, 1466, 1056, 969, 722 cm⁻¹

(2) Using the compound obtained in the above (1), the reaction was carried out in the same manner as Example 20 (5) to afford (R)-(E)-19-bromononadec-6-en-5-ol.

¹H-NMR(CDCl₃, 300 MHZ) δ ppm: 0.90 (t, J=6.8 Hz, 3H), 1.20-1.61 (m, 24H), 1.79-1.91 (m, 2H), 1.97-2.07 (m, 2H), 3.41 (t, J=6.8 Hz, 2H), 3.99-4.08 (m, 1H), 5.40-5.49 (m, 1H), 5.57-5.69 (m, 1H)

IR (neat): 3368, 2925, 2854, 2361, 1466, 1385 cm⁻¹

(3) Using the compound obtained in the above (2), the reaction was carried out in the same manner as Example 1 (6) to afford the title compound.

¹H-NMR (DMSO-d₆, 300 MHz) δ ppm: 0.78-0.96 (m, 3H), 1.10-1.61 (m, 26H), 1.88-2.03 (m, 2H), 2.31-2.42 (m, 2H), 3.78-3.90 (m, 1H), 4.49 (d, J=4.5 Hz, 1H), 5.30-5.54 (m, 2H)

IR (KBr): 3386, 2958, 2920, 2851, 1669, 1472, 1186, 1082, 1056, 965, 897, 803, 720, 614, 570, 524, 432 cm⁻¹

### Example 22

Sodium (R)-3-(10-hydroxytetradec-8-ynylsulfanyl) propane-1-sulfonate (Compound No. 19)

(1) Sodium hydride (153 mg, 60% dispersion in mineral oil, 3.82 mmol) was added to a solution of the compound obtained in Example 11 (1) (700 mg, 1.74 mmol), 3-mercapto-1-propanol (224 μL, 2.60 mmol) and sodium iodide (30 mg, 0.20 mmol) in THF (9.0 mL) and mixture was stirred at 45° C. for 7 hours. To the resulting solution was added a saturated aqueous NH₄Cl solution (50 mL) and the mixture was extracted with AcOEt (50 mL×2). The organic layer was washed with water (50 mL) and brine (50 mL), dried over anhydrous magnesium sulfate and concentrated. The resulting crude product was purified by column chromatography to afford (R)-3-[10-(tert-butyldimethylsilanyloxy)tetradec-8-ynylsulfanyl]propan-1-ol (650 mg).

¹H-NMR (CDCl₃, 300 MHz) δ ppm: 0.10 (s, 3H), 0.12 (s, 3H), 0.84-0.97 (m, 3H), 0.90 (s, 9H), 1.25-1.70 (m, 16H), 1.80-1.91 (m, 2H), 2.18 (dt, J=1.9, 6.9 Hz, 2H), 2.53 (t, J=7.3 Hz, 2H), 2.64 (t, J=7.1 Hz, 2H), 3.77 (t, J=6.1 Hz, 2H), 4.31 (tt, J=6.5, 1.9 Hz, 1H)

IR (neat): 3231, 2930, 2857, 1630, 1462, 1387, 1361, 1342, 1294, 1251, 1152, 1062, 1036, 938, 837, 777, 668, 629, 596 cm⁻¹

(2) Using the compound obtained in the above (1), the reaction was carried out in the same manner as Example 1 (3) to afford (R)-[10-(3-bromopropylsulfanyl)-1-butyldec-2-ynyloxy]-tert-butyldimethylsilane.

¹H-NMR (CDCl₃, 300 MHz) δ ppm: 0.10 (s, 3H), 0.12 (s, 3H), 0.86-0.94 (m, 3H), 0.90 (s, 9H), 1.23-1.69 (m, 16H), 2.06-2.22 (m, 4H), 2.51 (t, J=7.4 Hz, 2H), 2.66 (t, J=6.9 Hz, 2H), 3.52 (t, J=6.5 Hz, 2H), 4.31 (tt, J=6.5, 1.9 Hz, 1H)

IR (neat): 3118, 2930, 2857, 1463, 1402, 1361, 1250, 1152, 1109, 1083, 1005, 938, 837, 777, 668, 565 cm⁻¹

(3) Using the compound obtained in the above (2), the reaction was carried out in the same manner as Example 1 (4) to afford (R)-14-(3-bromopropylsulfanyl)tetradec-6-yn-5-ol.

¹H-NMR (CDCl₃, 300 MHz) δ ppm: 0.92 (t, J=7-1 Hz, 3H), 1.23-1.75 (m, 16H), 2.04-2.24 (m, 4H), 2.52 (t, J=7.4 Hz, 2H), 2.66 (t, J=6.9 Hz, 2H), 3.52 (t, J=6.5 Hz, 2H), 4.30-4.39 (m, 1H)

IR (neat): 3231, 2930, 2857, 2230, 1630, 1461, 1434, 1384, 1333, 1294, 1242, 1148, 1104, 1036, 728, 629, 596, 563 cm⁻¹

(4) Using the compound obtained in the above (3), the reaction was carried out in the same manner as Example 1 (6) to afford the title compound.

¹H-NMR (DMSO-d₆, 300 MHz) δ ppm: 0.86 (t, J=7.1 Hz, 3H), 1.20-1.58 (m, 16H), 1.73-1.85 (m, 2H), 2.16 (dt, J=2.0, 6.7 Hz, 2H), 2.42-2.57 (m, 6H), 4.09-4.18 (m, 1H), 5.07 (d, J=5.6 Hz, 1H)

IR (KBr): 3508, 3360, 2927, 2857, 1654, 1454, 1278, 1250, 1221, 1206, 1177, 1152, 1100, 1059, 1010, 891, 847, 811, 778, 748, 716, 609, 541, 526, 455 cm⁻¹

### Example 23

Sodium (R)-(Z)-3-(10-hydroxytetradec-8-enylsulfanyl) propane-1-sulfonate (Compound No. 47)

Quinoline (18 μL) was added dropwise at room temperature, under hydrogen atmosphere, to a suspension of Pd—CaCO₃ (40 mg) in MeOH (5.0 mL) and the mixture was stirred at that temperature for 45 minutes. To the reaction mixture was added dropwise at room temperature a solution of the compound obtained in Example 22 (100 mg, 0.259 mmol) in MeOH (1.0 mL) and the mixture was stirred at that temperature for about 1.5 hours until absorption of hydrogen gas ceased. The mixture was filtered through a celite pad and concentrated. The resulting crude product was purified by column chromatography to afford the title compound (90 mg).

¹H-NMR (DMSO-d₆, 300 MHz) δ ppm: 0.85 (t, J=6.7 Hz, 3H), 1.14-1.56 (m, 16H), 1.72-1.85 (m, 2H), 1.93-2.09 (m, 2H), 2.41-2.57 (m, 6H), 4.10-4.27 (m, 1H), 4.47 (d, J=4.7 Hz, 1H), 5.21-5.35 (m, 2H)

IR (KBr): 3330, 2924, 2852, 1656, 1467, 1378, 1203, 1080, 1057, 820, 752, 602, 528, 419 cm⁻¹

### Example 24

Sodium (R)-3-(10-hydroxytetradec-8-ynyloxy)propane -1-sulfonate (Compound No. 21)

(1) To a suspension of sodium hydride (324 mg, oil free, 13.5 mmol) in DMF (N,N-dimethylformamide)(13.0 mL) was added 1,3-propanediol (1.09 mL, 15.0 mmol) at 0° C. and the mixture was stirred at that temperature for 10 minutes and at room temperature for 10 minutes. To the resulting solution were added at 0° C. a solution of the compound obtained in Example 11 (1) (1.21 g, 3.00 mmol) in DMF (2.0 mL) and sodium iodide (45 mg) and the mixture was stirred at room temperature for 7 hours. To the resulting solution was added a saturated aqueous NH₄Cl solution (70 ml) and the mixture was extracted with mixed solvent of AcOEt and Hexane (3:1) (70 mL×2). The organic layer was washed with water (50 mL×3) and brine (50 mL), dried over an hydrous magnesium sulfate and concentrated. The resulting crude product was purified by column chromatography to afford (R)-3-[10-(tert-butyldimethylsilanyloxy)tetradec-8-yny loxy]propan-1-ol (660 mg).

¹H-NMR (CDCl₃, 300 MHz) δ ppm: 0.10 (s, 3H), 0.12 (s, 3H), 0.85-0.94 (m, 3H), 0.90 (s, 9H), 1.24-1.67 (m, 16H), 1.75-1.87 (m, 2H), 2.18 (dt, J=1.9, 6.9 Hz, 2H), 3.43 (t, J=6.6 Hz, 2H), 3.61 (t, J=5.7 Hz, 2H), 3.78 (t, J=5.5 Hz, 2H), 4.31 (tt, J=6.6, 1.9 Hz, 1H)

IR (neat): 3119, 2930, 2858, 1463, 1401, 1251, 1151, 1115, 1084, 938, 837, 777, 667 cm⁻¹

(2) Using the compound obtained in the above (1), the reaction was carried out in the same manner as Example 1 (3) to afford (R)-[10-(3-bromopropoxy)-1-butyldec-2-ynyloxy]-tert-butyldimethylsilane.

¹H-NMR (CDCl₃, 300 MHz) δ ppm: 0.10 (s, 3H), 0.12 (s, 3H), 0.86-0.94 (m, 3H), 0.90 (s, 9H), 1.23-1.67 (m, 16H), 2.04-2.14 (m, 2H), 2.18 (dt, J=1.9, 6.9 Hz, 2H), 3.42 (t, J=6.6 Hz, 2H), 3.47-3.56 (m, 4H), 4.31 (tt, J=6.5, 1.9 Hz, 1H)

IR (neat): 3228, 2931, 2858, 1630, 1463, 1362, 1294, 1255, 1212, 1150, 1116, 1081, 1036, 938, 837, 778, 666, 596 cm⁻¹

(3) Using the compound obtained in the above (2), the reaction was carried out in the same manner as Example 1 (4) to afford (R)-14-(3-bromopropoxy)tetradec-6-yn-5-ol.

¹H-NMR(CDCl₃, 300 MHz) δ ppm: 0.92 (t, J=7.1 Hz, 3H), 1.22-1.78 (m, 16H), 2.04-2.14 (m, 2H), 2.21 (dt, J=1.9, 7.0 Hz, 2H), 3.42 (t, J=6.6 Hz, 2H), 3.48-3.56 (m, 4H), 4.30-4.39 (m, 1H)

IR (neat): 3400, 3118, 2933, 2859, 1673, 1466, 1401, 1286, 1257, 1212, 1148, 1116, 1037, 892, 768, 654, 573 cm⁻¹

(4) Using the compound obtained in the above (3), the reaction was carried out in the same manner as Example 1 (6) to afford the title compound.

¹H-NMR (DMSO-d₆, 300 MHz) δ ppm: 0.86 (t, J=7.1 Hz, 3H), 1.20-1.58 (m, 16H), 1.70-1.82 (m, 2H), 2.12-2.21 (m, 2H), 2.37-2.45 (m, 2H), 3.28-3.40 (m, 4H), 4.09-4.19 (m, 1H), 5.08 (d, J=5.4 Hz, 1H)

IR (KBr): 3360, 2932, 2857, 2799, 2230, 1656, 1468, 1376, 1210, 1192, 1117, 1055, 901, 793, 744, 621, 555, 530, 482 cm⁻¹

### Example 25

Lithium (R)-(Z)-15-hydroxynonadec-13-ene-1-sulfonate (Compound No. 37)

To a solution of the compound obtained in Example 3 (100 mg, 0.254 mmol) in EtOH (5.0 mL) was added dropwise, under argon stream, a solution of alcoholic hydrogen chloride (1.0 mL, 0.5M) and the mixture was stirred at room temperature for 2 hours. The resulting precipitate was filtered out. To the filtrate was added an aqueous solution of LiOH (1.0 mL, 1.0M), and then the mixture was stirred at room temperature for 2 hours and concentrated. The resulting crude product was purified by resin(HP-20, Nippon Rensui) to afford the title compound (96 mg).

¹H-NMR (DMSO-d₆, 300 MHz) δ ppm: 0.85 (t, J=6.7 HZ, 3H), 1.12-1.59 (m, 26H), 1.94-2.05 (m, 2H), 2.30-2.39 (m, 2H), 4.15-4.28 (m, 1H), 4.47 (d, J=4.5 Hz, 1H), 5.21-5.35 (m, 2H)

IR (KBr): 3342, 3014, 2958, 2932, 2922, 2848, 1656, 1464, 1407, 1291, 1222, 1186, 1077, 962, 872, 803, 726, 621, 566, 543, 472 cm⁻¹

### Example 26

Potassium (R)-(Z)-15-hydroxynonadec-13-ene-1-sulfonate (Compound No. 35)

The reaction was carried out substantially in the same manner as Example 25, but using an aqueous solution of KOH instead of an aqueous solution of LiOH, to afford the title compound.

¹H-NMR (DMSO-d₆, 300 MHz) δ ppm: 0.85 (t, J=6.6 Hz, 3H), 1.15-1.60 (m, 26H), 1.93-2.07 (m, 2H), 2.30-2.39 (m, 2H), 4.13-4.25 (m, 1H), 4.47 (d, J=4.5 Hz, 1H), 5.21-5.35 (m, 2H)

IR (KBr): 3347, 3007, 2924, 2918, 2852, 1470, 1379, 1200, 1191, 1053, 1020, 794, 721, 609, 550, 530 cm⁻¹

### Example 27

Ammonium (R)-(Z)-15-hydroxynonadec-13-ene-1-sulfonate (Compound No. 38)

The reaction was carried out substantially in the same manner as Example 25, but using 28% aqueous ammonia instead of an aqueous solution of LiOH, to afford the title compound.

¹H-NMR (CD₃OD, 300 MHz) δ ppm: 0.91 (t, J=6.8 Hz, 3H), 1.18-1.66 (m, 24H), 1.70-1.85 (m, 2H), 1.98-2.16 (m, 2H), 2.72-2.84 (m, 2H), 4.31-4.43 (m, 1H), 5.26-5.51 (m, 2H)

IR (neat): 3206, 2924, 2853, 1652, 1466, 1170, 1084, 1042, 792, 756, 722, 609, 529 cm⁻¹

### Example 28

(R)-(Z)-15-hydroxynonadec-13-ene-1-sulfonic acid [tris(hydroxymethyl)methyl]amine salt (Compound No. 39)

The reaction was carried out substantially in the same manner as Example 25, but using tris(hydroxymethyl)aminomethane instead of an aqueous solution of LiOH, to afford the title compound.

¹H-NMR (CD₃OD, 300 MHZ) δ ppm: 0.91 (t, J=6.8 Hz, 3H), 1.23-1.64 (m, 24H), 1.70-1.85 (m, 2H), 1.98-2.14 (m, 2H), 2.73-2.83 (m, 2H), 3.64 (s, 6H), 4.30-4.43 (m, 1H), 5.26-5.37 (m, 1H), 5.38-5.50 (m, 1H)

IR (KBr): 3340, 3232, 2919, 2851, 1630, 1516, 1468, 1294, 1188, 1051, 793, 756, 722, 610, 531 cm⁻¹

### Example 29

(R)-(Z)-15-hydroxynonadec-13-ene-1-sulfonic acid (L)-Lysine salt (Compound No. 40)

The reaction was carried out substantially in the same manner as Example 25, but using (L)-Lysine instead of an aqueous solution of LiOH, to afford the title compound.

¹H-NMR (CD₃OD, 300 MHZ) δ ppm: 0.91 (t, J=6.5 Hz, 3H), 1.16-1.91 (m, 32H), 1.98-2.14 (m, 2H), 2.73-2.82 (m, 2H), 2.88-2.97 (m, 2H), 3.50-3.58 (m, 1H), 4.30-4.42 (m, 1H), 5.24-5.36 (m, 1H), 5.38-5.50 (m, 1H)

IR (KBr): 2923, 1560, 1508, 1466, 1407, 1323, 1170, 1044, 900, 863, 797, 728, 668, 611, 538, 472, 459, 435, 428, 418 cm⁻¹

### Example 30

(R)-(Z)-15-Acetoxynonadec-13-ene-1-sulfonic acid amide (Compound No. 45)

A solution of the compound obtained in Example 19 (150 mg, 0.325 mmol) in DMF (0.2 mL) was added at 0° C. to thionyl chloride (0.20 mL) and then the mixture was stirred at that temperature for 2 hours. To the resulting solution was added water (20 mL) and then the mixture was extracted with AcOEt (30 mL×2). The organic layer was washed with water (30 mL), dried over anhydrous magnesium sulfate and concentrated. Anhydrous ammonia was bubbled into a solution of the resulting crude sulfonylchloride in CH₂Cl₂ (2 mL) at room temperature for 30 minutes. The resulting precipitate was filtered out and the filtrate was concentrated. The resulting crude product was purified by silica gel column chromatography to afford the title compound (40 mg).

¹H-NMR (CDCl₃, 300 MHZ) δ ppm: 0.89 (t, J=7.0 Hz, 3H), 1.18-1.73 (m, 24H), 1.79-1.93 (m, 2H), 1.96-2.24 (m, 5H), 3.07-3.16 (m, 2H), 4.56 (bs, 2H), 5.23-5.34 (m, 1H), 5.48-5.59 (m, 2H)

IR (neat): 3255, 3014, 2925, 2854, 1736, 1556, 1466, 1401, 1371, 1332, 1241, 1149, 1084, 1019, 953, 723, 573, 498 cm⁻¹

### Example 31

(R)-(Z)-15-Hydroxynonadec-13-ene-1-sulfonic acid amide (Compound No. 46)

Sodium methoxide (27 mg, 0.500 mmol) was added at room temperature to a solution of the compound obtained in Example 30 (40 mg, 0.0991 mmol) in MeOH (2.0 mL) and the mixture was stirred at that temperature overnight. To the resulting mixture was added water, and the mixture was extracted with AcOEt (30 mL×2), dried over anhydrous magnesium sulfate and concentrated. The resulting crude product was purified by silica gel column chromatography to afford the title compound (27 mg).

¹H-NMR(CDCl₃, 300 MHz) δ ppm: 0.91 (t, J=6.9 Hz, 3H), 1.20-1.65 (m, 24H), 1.80-1.93 (m, 2H), 1.98-2.18 (m, 2H), 3.07-3.15 (m, 2H), 4.37-4.56 (m, 3H), 5.31-5.42 (m, 1H), 5.43-5.54 (m, 1H)

IR (KBr): 3359, 2919, 2848, 1736, 1686, 1656, 1543, 1462, 1339, 1302, 1284, 1140, 1054, 899, 790, 724, 644, 591, 518, 489, 418 cm⁻¹

### Example 32 (R)-(Z)-15-Hydroxynonadec-13-ene-1-sulfonic acid methyl ester (Compound No. 72)

To a solution of the compound obtained in Example 3 (100 mg, 0.254 mmol) in EtOH (5.0 mL) was added dropwise a solution of alcoholic hydrogen chloride (1.0 mL, 0.5M) at room temperature, and the mixture was stirred at that temperature for 2 hours. The resulting precipitate was filtered out. To the filtrate was added (trimethylsilyl)diazomethane (1.0 mL, 2.0M in THF solution) at room temperature, and then stirred at room temperature for 2 hours. The resulting reaction mixture was poured into water and the mixture was extracted with AcOEt (50 mL×2). The organic layer was washed with brine (50 mL), dried over anhydrous magnesium sulfate and concentrated. The resulting crude product was purified by silica gel column chromatography to afford the title compound (20 mg).

¹H-NMR (CDCl₃, 300 MHz) δ ppm: 0.91 (t, J=6.8 Hz, 3H), 1.19-1.66 (m, 24H), 1.78-1.92 (m, 2H), 1.98-2.18 (m, 2H), 3.05-3.14 (m, 2H), 3.89 (s, 3H), 4.37-4.48 (m, 1H), 5.32-5.41 (m, 1H), 5.43-5.54 (m, 1H)

IR (KBr): 3376, 2920, 2851, 1585, 1510, 1471, 1412, 1205, 1187, 1080, 1050, 863, 806, 721, 610, 528, 428 cm⁻¹

### Test Example 1

Test for Elastase Production by fMLP (N-formyl-Met-Leu-Phe)Stimulation

Rat neutrophils preparation was obtained 15-18 hours after intraperitoneal injection of a 1% sterile casein solution in saline (120 mL/kg). Cells were harvested by peritoneal lavage after the decapitation. The lavage fluid was ice-cold PBS (Phosphate-Buffered Saline). Peritoneal exudates were pooled, centrifuged and suspended in HBSS (Hanks' Balanced Salt Solution) at 1×10⁷ cells/mL. Cytochalasin B (final concentration: 5 μg/mL) were added to prime the cells. The cells were added into a 96-well culture plate (190 μL/well) and then the compounds of the present invention at various concentrations (10⁻⁷ to 3×10⁻⁵ M) were added and incubated at 37° C. in an atmosphere of 5% CO₂ in air. After 10 minutes, fMLP (20 μM, 10 μL) was added, while 10 μL of an HBSS solution containing 0.4% ethanol was added to the group to which no fMLP was added. After gently stirring, cells were incubated for further 10 minutes. The reaction was stopped on ice, and an incubated supernatant was recovered by centrifugation.

Assay of Elastase Activity in an Incubated Supernatant

Elastase activity in the incubated supernatant was measured using a specific elastase substrate, N-succinyl-L-alanyl-L-alanyl-L-proline-valine-MCA (Peptide Institue, Inc., Osaka), 0.12 mM in 50 mM Tris-HCl (pH 8.0). Fifty microliter of an incubated supernatant was added to the substrate solution (50 μL) and incubated at 37° C. for 30 minutes. Elastase activity was assayed at a wavelength of 360 nm at Excitation and 480 nm at Emission.

Elastase release-inhibiting activity (inhibition ratio) was calculated according to the following equation:
Inhibition ratio (%) {1−(A−C)/(B−C)}×100
wherein A stands for a fluorescence intensity when fMLP (1 μM) was added; B stands for a fluorescence intensity when fMLP (1 μM) and the present compound were added; and C stands for a fluorescence intensity when fMLP (1 μM) was not added.

Inhibitory concentration of 50% (IC₅₀ Value) of the compound of the invention was calculated with a concentration-inhibition ratio curve. The results are shown in Table 1.

TABLE 1

| Test compound | IC₅₀ Value (μM) |
|---|---|
| Compound 23 | 9.67 |
| Compound 33 | 15.0 |

In the above Table, Compounds 23 and 33 correspond to the compounds of the Examples. The above results demonstrate that the compound of the present invention has a potent inhibiting activity in elastase production.

### Test Example 2

Effect on the Infarct Volume in Rat Transient MCA Occlusion (t-MCAo) Model.

Methods

Adult male Wistar rats (200-250 g) were anesthetized with 2% halothane in air. The right internal carotid artery (ICA) was carefully dissected. A silicon-coated suture (18 mm-long) was inserted into the ICA. Body temperature was maintained at 37° C. with a heating pad. After surgery, anesthesia was discontinued, and ischemic animal exhibited severe hemiparesis in the upper extremities. After 1 hour of MCA occlusion, the thread was removed to allow reperfusion of the ischemic area. Rats were received intravenously 1 hour-infusion of vehicle (10% of HP-β-CD) or compound 33 dissolved in vehicle immediately after reperfusion.

To measure infarct volume, rats were killed at 71 hours of reperfusion. Brains were perfused transcardially with physiological saline, and removed from skulls, cut into 2-mm coronal sections. The slices were immersed in 2% triphenyltetrazolium chloride (TTC) solution at 37° C. for 30 minutes. All values were presented as mean±SEM. For statistical analyses, Dunnett's multiple-range test was used.

Results

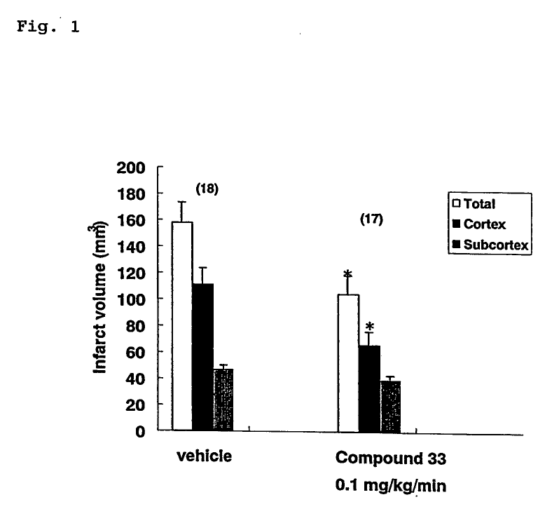

Compound 33 (0.1 mg/kg/min) dissolved in 10% of HP-β-CD was continuously administered for 1 hour from immediately after reperfusion. Compound 33 significantly reduced the total and cortex infarct volume as compared with vehicle-treated group at a dose of 0.1 mg/kg/min, 1 hour (FIG. 1). This result indicates that compound 33 has a neuroprotective efficacy against ischemic brain damage.

### INDUSTRIAL APPLICABILITY

The hydroxyeicosenoic acid analog according to the invention has a potent elastase release-inhibiting activity and it is then useful as an elastase release inhibitor.

Elastase is known to be involved in pathology of certain diseases such as pulmonary emphysema, respiratory distress syndrome of adults, idiopathic pulmonary fibrosis, cystic pulmonary fibrosis, chronic interstitial pneumonia, chronic bronchitis, chronic sinopulmonary infection, diffuse panbronchiolitis, bronchiectasis, asthma, pancreatitis, nephritis, hepatic insufficiency, chronic rheumatism, arthrosclerosis, osteoarthritis, psoriasis, periodontitis, atherosclerosis, rejection against organ transplantation, premature amniorrhexis, hydroa, shock, sepsis, systemic lupus erythematosus, Crohn's disease, disseminated intravenous coagulation, cerebral infarction, cardiac disorders, ischemic reperfusion disorders observed in renal diseases, cicatrization of corneal tissues, spondylitis, and etc.

The elastase release inhibitor according to the invention is therefore useful as a therapeutic or preventive agent for the above-mentioned diseases.

### List of the Prior Art Literature(s)

1. WO 01/34548
1. WO 01/34550
1. WO 01/34551

1. A hydroxyfattysulfonic acid analog represented by Formula (I): 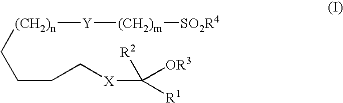
wherein
X is an ethylene group, a vinylene group or an ethynylene group; Y is an ethylene group, a vinylene group, an ethynylene group, OCH₂ or S(O)pCH₂, wherein p is 0, 1 or 2; m is an integer of 1 to 5 inclusive; n is an integer of 0 to 4 inclusive;
R¹ is a C₁₋₆ alkyl group, a C₃₋₈ cycloalkyl group, a C₁₋₄ alkyl group substituted with a C₃₋₈ cycloalkyl group, a C₁₋₄ alkyl group substituted with an aryl group or a C₁₋₄ alkyl group substituted with an aryloxy group; R² is a hydrogen atom or a methyl group; R¹ and R² together with the carbon atom to which they are attached may form a C₃₋₈ cycloalkyl group; R³ is a hydrogen atom or a C₂₋₈ acyl group; R⁴ is OR⁵ or NHR⁶, wherein R⁵ is a hydrogen atom, a C₁₋₄ alkyl group, an alkali metal, an alkaline earth metal or an ammonium group and R⁶ is a hydrogen atom or a C₁₋₄ alkyl group; or
a pharmaceutically acceptable salt or a hydrate thereof.

2. The hydroxyfattysulfonic acid analog of Formula (I) according to claim 1 wherein X is a vinylene group or an ethynylene group, Y is an ethylene group, a vinylene group, an ethynylene group, OCH₂ or SCH₂, R¹ is a C₁₋₈ alkyl group or a C₃₋₈ cycloalkyl group, R² is a hydrogen atom or a methyl group, R³ is a hydrogen atom, R⁴ is OR⁵ group and the sum of m and n is an integer of from 4 to 8, or a pharmaceutically acceptable salt or the hydrate thereof.

3. The hydroxyfattysulfonic analog of Formula (I) according to claim 1 wherein the compound is sodium (R)-(4Z, 13Z)-15-hydroxynonadeca-4,13-diene-1-sulfonate or sodium (R)-(Z)-15-hydroxynonadec-13-ene-1-sulfonate

4. An elastease-inhibiting composition which comprises a hydroxyfattysulfonic acid analog represented by the Formula (I): 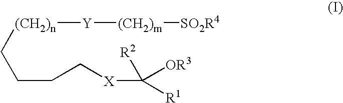
wherein
X is an ethylene group, a vinylene group or an ethynylene group; Y is an ethylene group, a vinylene group, an ethynylene group, OCH₂ or S(O)pCH₂, wherein p is 0, 1 or 2; m is an integer of 1 to 5 inclusive; n is an integer of 0 to 4 inclusive;
R¹ is a C₁₋₈ alkyl group, a C₃₋₈ cycloalkyl group, a C₁₋₄ alkyl group substituted with a C₃₋₈ cycloalkyl group, a C₁₋₄ alkyl group substituted with an aryl group or a C₁₋₄ alkyl group substituted with an aryloxy group; R² is a hydrogen atom or a methyl group; R¹ and R² together with the carbon atom to which they are attached may form a C₃₋₈ cycloalkyl group; R³ is a hydrogen atom or a C₂₋₈ acyl group; R⁴ is OR⁵ or NHR⁶, wherein R⁵ is a hydrogen atom, a C₁₋₄ alkyl group, an alkali metal, an alkaline earth metal or an ammonium group and R⁶ is a hydrogen atom or a C₁₋₄ alkyl group; or
a pharmaceutically acceptable salt or a hydrate thereof and a pharmaceutically acceptable carrier.
